(19) United States
(12) Patent Application Publication
(10) Pub. No.: US 2025/0099086 A2
(43) Pub. Date: Mar. 27, 2025

(54) NANOFIBER COLLECTION DEVICES

(71) Applicant: Board of Regents of the University of Nebraska, Lincoln, NE (US)

(72) Inventors: Jingwei Xie, Omaha, NE (US); Johnson Vitharikunnil John, Los Angeles, CA (US)

(21) Appl. No.: 18/194,664

(22) Filed: Apr. 3, 2023

Related U.S. Application Data
(60) Provisional application No. 63/348,541, filed on Jun. 3, 2022.

(51) Int. Cl. A61B 10/00; A61F 13/15; A61F 13/36; A61L 15/26

(52) U.S. Cl. CPC A61B 10/0045; A61F 13/15723; A61F 13/36; A61L 15/26; A61B 2010/0061; A61B 2562/162; A61L 2400/12

(57) ABSTRACT

Sample collection devices are provided as well as methods of use thereof and methods of making.

This application claims priority under 35 U.S.C. § 119 (e) to U.S. Provisional Patent Application No. 63/348,541, filed Jun. 3, 2022. The foregoing application is incorporated by reference herein.

This invention was made with government support under Grant No. R01 GM123081 awarded by the National Institutes of Health and Grant No. W81XWH2010207 awarded by the Department of Defense. The government has certain rights in the invention.

### FIELD OF THE INVENTION

This application relates to the fields of nanofiber structures. More specifically, this invention provides nanofiber collection devices, methods of synthesizing, and methods of use thereof.

### BACKGROUND OF THE INVENTION

Several publications and patent documents are cited throughout the specification in order to describe the state of the art to which this invention pertains. Each of these citations is incorporated herein by reference as though set forth in full.

Collections of cell/tissue and microorganism samples are critical for early diagnosis of many diseases like cancer and pathogenic infections, potentially resulting in effective interventions and prevention of disease transmission (Watts G, Lancet (2018) 391:2593-2594; Etzioni, et al., Nat. Rev. Cancer (2003) 3:243-252; Chu, et al., N. Engl. J. Med. (2020) 383:185-187; Ashford, et al., Emerg. Infect. Dis. (2003) 9:515-519). For most parts of the Western world, population-based endoscopic screening is neither feasible nor cost-effective due to the low prevalence of Barrett's and gastric premalignant conditions (Lochhead, et al., JAMA Intern. Med. (2015) 175:159-160; Spechler, et al., Rev. Gastroenterol. Disord. (2002) 2: S25-S29). Thus, prescreening of the general population with minimally or non-invasive tests to identify individuals at heightened risk in whom further endoscopic assessment should be undertaken is desirable. Towards this end, Cytosponge™, a cell collection device, was developed for the collection and retrieval of surface cells in the esophagus and is currently being investigated in clinical studies (Fitzgerald, et al., Lancet (2020) 396:333-344; Offman, et al., BMC Cancer (2018) 18:784; Benaglia, et al., Gastroenterology (2013) 144:62; di Pietro, et al., Gastroenterology (2015) 148:912-923; Katzka, et al., Clin. Gastroenterol. Hepatol. (2015) 13:77-83; Swart, et al., Eclinical Medicine (2021) 37:100969). The Cytosponge™ is composed of a compressed 30-mm polyurethane sponge attached to a string encased in an ingestible gelatin capsule (Offman, et al., BMC Cancer (2018) 18:784; Benaglia, et al., Gastroenterology (2013) 144:62; di Pietro, et al., Gastroenterology (2015) 148:912-923; Katzka, et al., Clin. Gastroenterol. Hepatol. (2015) 13:77-83; Swart, et al., Eclinical Medicine (2021) 37:100969). However, the Cytosponge™ retrieval process has caused some serious issues arising from the detachment of the sponge from the removal string during withdrawal from the patient's esophagus and occasionally causing minor pharyngeal bleeds (Januszewicz et al., Clin. Gastroenterol. Hepatol. (2019) 4:647). In addition, this device is only used for sampling the esophageal mucosa, rather than collecting fluids from stomach and duodenum due to its rigid, porous structure. Similarly, EsophaCap™ a polyurethane derived expandable sponge on a string has also been used to collect the samples from the esophagus in minimally invasive way. The immunohistochemistry of collected cells has been used to identify the stage of esophagus adenosarcoma like the Cytosponge™ based analysis (Zhou, et al., Clin. Exp. Gastroenterol. (2019) 12:219-229; Wang, et al., Clin. Cancer Res. (2019) 25:2127-2135). The shortcomings of these collection devices show that improved collection capsules are needed.

### SUMMARY OF THE INVENTION

In accordance with the instant invention, sample collection devices are provided along with methods of producing the sample collection devices and methods of use. In certain embodiments, the method for producing a sample collection device comprises a) fixing (e.g., thermally and/or chemically) at least one point of a nanofiber mat (e.g., a side of a nanofiber mat), b) expanding the nanofiber mat by exposing the nanofiber mat to gas bubbles (optionally by more than one exposure), thereby producing an expanded nanofiber structure, and c) attaching the expanded nanofiber structure to an end of a string, thereby producing the sample collection devices. In certain embodiments, the string is attached to the nanofiber structure prior to expansion. In certain embodiments, the nanofiber mat comprises electrospun nanofibers. In certain embodiments, the expanded nanofiber structure has a rounded geometry (e.g., spherical). In certain embodiments, the expanded nanofiber structure has a rectangular geometry (e.g., rectangular or cubic). In certain embodiments, the gas bubbles are generated as a product of a chemical reaction (e.g., the hydrolysis of sodium borohydride). In certain embodiments, the gas bubbles are generated by a physical releasing (e.g., depressurized CO₂). In certain embodiments, the nanofiber mat is expanded by exposing the nanofiber mat to a subcritical fluid (e.g., subcritical CO₂) and depressurizing (e.g., within a container). In certain embodiments, the nanofiber mat comprises a plurality of aligned nanofibers, random nanofibers, and/or entangled nanofibers. In certain embodiments, the method further comprises synthesizing the nanofiber mat by electrospinning prior to step a). In certain embodiments, the method further comprises cutting and/or trimming the nanofiber mat (e.g., prior to step b) into a desired shape (e.g., semicircular shape). In certain embodiments, the nanofiber mat is frozen (e.g., with liquid nitrogen) prior to cutting and/or trimming. In certain embodiments, the nanofiber mat comprises polycaprolactone (PCL) and, optionally, a poloxamer (e.g., poloxamer 407 or poloxamer 188). In certain embodiments, the method further comprises coating the expanded nanofiber structure (e.g., before and/or after attachment of the string) with a substance such as a hydrogel (e.g., gelatin, gelatin methacryloyl(GelMA), and/or chitosan). In certain embodiments, the nanofibers/nanofiber structure are further decorated/conjugated with biomarkers (e.g., antibodies) for diagnosis of certain diseases (e.g., pathogen infection, gastric cancer, esophagus cancer). In certain embodiments, the nanofibers/nanofiber structure is combined with soft electronics (e.g., for in situ diagnosis). In certain embodiments, the method further comprises crosslinking the expanded nanofiber structure and/or coating substance (e.g., hydrogel). In certain embodiments, the crosslinking is achieved by contacting and/or exposure to a crosslinker (e.g., glutaraldehyde) or a thermal treatment. In certain embodiments, the string is attached to the expanded nanofiber structure by an adhesive (e.g., an epoxy) or by physical attachment (e.g., knotting). In certain embodiments, the methods further comprise encapsulating the expanded nanofiber structure in a capsule (e.g., a gelatin capsule).

In accordance with the instant invention, sample collection devices comprising an expanded nanofiber structure and a string are provided. In certain embodiments, the expanded nanofiber structure is attached to a terminus of the string. In certain embodiments, the sample collection device is produced by a method of the instant invention.

In accordance with the instant invention, methods of collecting a sample from a subject are provided. In certain embodiments, the method comprises contacting a sample collection device with a site in the subject. In certain embodiments, the method comprises administering the sample collection device to the subject. In certain embodiments, the sample is collected from the site via the expanded nanofiber structure. In certain embodiments, the sample is a biological sample. In certain embodiments, the sample comprises a bacteria, a virus, a cell, a tissue, and/or a fluid. In certain embodiments, the sample comprises a nucleic acid and/or a polypeptide. In certain embodiments, the method further comprises retrieving the sample collection device from the subject (e.g., by using and/or pulling the string). In certain embodiments, the method further comprises analyzing (e.g., via microscopy) the sample for a disease or disorder (e.g., Barret Esophagus, acid reflux (gastric reflux), SARS-COVID-19, Helicobacter pylori, cancer (e.g., gastric cancer, esophageal cancer, etc.), and the like) or detecting in the sample a disease or disorder (e.g., by detecting a biomarker).

### BRIEF DESCRIPTION OF THE DRAWINGS

FIG. 1 provides a schematic of the swallowable, re-expandable, ultra-absorbable, and retrievable nanofiber capsule being used for biological specimen collection. A schematic illustration is provided of the nanofiber capsule on a string in stomach, dissolution of the capsule at gastric site, and re-expansion and retrieval of the nanofiber object. The possible sample collection sites like duodenal, gastric, esophagus and oropharyngeal sites are also depicted.

FIGS. 2A-2L show the fabrication of swallowable, re-expandable, ultra-absorbable, and retrievable nanofiber capsules. FIGS. 2A and 2B: Schematic illustration of the fabrication of cuboid-shaped nanofiber objects expanded from a 2D nanofiber mat. FIGS. 2C-2E: Photographs and SEM images of a cuboid-shaped nanofiber object. FIGS. 2F-2G: Schematic illustration of the fabrication of a sphere-shaped nanofiber object transformed from a 2D nanofiber mat. FIGS. 2H-2J: Photographs and SEM images of a sphere-shaped nanofiber object. The small rectangle in FIG. 2I represents the thermally welded region. FIG. 2K: Photograph shows a nanofiber sphere attached with a string. FIG. 2L: A dissolvable gelatin capsule encapsulated with a compressed nanofiber sphere on a string. The arrows indicate the attached string.

FIG. 3A provides a graph of the dissolution time of gelatin capsules encased with different nanofiber objects at different pHs (n=3). Cuboid: capsules encased with cuboid-shaped nanofiber objects, Sphere: capsules encased with sphere-shaped nanofiber objects, Unexpanded square: capsules encased with unexpanded square nanofiber membranes, and Unexpanded semicircle: capsules encased with unexpanded semicircular nanofiber membranes. FIG. 3B provides a graph of water absorption capacity of cuboid- and sphere-shaped nanofiber objects and square and semicircular nanofiber membranes with increasing the incubation time.

FIGS. 4A-4E show the bacteria collection. FIG. 4A: Photographs of the plates of MRSA colonies after recovery from cuboid- and sphere-shaped nanofiber objects and semicircular and square nanofiber membranes which were used for swabbing stock solutions of 10⁶, 10⁵, 10⁴, and 10³ CFU/ml. FIGS. 4B-4E: MRSA colony counts after recovery from cuboid- and sphere-shaped nanofiber objects and semicircular and square nanofiber membranes which were used for swabbing stock solutions of 10⁶, 10⁵, 10⁴, and 10³ CFU/ml.

FIGS. 5A-5C show SARS-Cov-2 detection and identification. FIG. 5A: Schematic representation from SARS-COV-2 detection using PCR and swabbing. FIG. 5B: Cycle threshold values at each SARS-COV-2 titer following swabbing. FIG. 5C: Converted SARS-COV-2 concentration in pfu/ml. *p<0.05, **p<0.01, ***p<0.001.

FIGS. 6A-6F show tissue collection from swine esophagus. FIG. 6A: Photographs showing the cell/tissue collection process from swine esophagus using sphere-shaped nanofiber objects. FIG. 6B: Photographs showing the cell/tissue collection process from swine esophagus using semicircular nanofiber membranes. FIG. 6C: Photograph showing the inside view of the swine esophagus. The dotted line indicates the inner wall of swine esophagus. FIG. 6D: Photograph showing the nanofiber sphere in the swine esophagus. Arrows indicate the nanofiber sphere and the attached string, respectively. FIGS. 6E and 6F: Representative hematoxylin and eosin (H&E) stained images of collected tissues from the swine esophagus.

FIGS. 7A and 7B show the mechanical properties of string attached nanofiber capsules. FIG. 7A: Tensile break force (string detachment force) of different nanofiber capsules related to displacement. FIG. 7B: Comparative representation of tensile break forces of different nanofiber objects like, cuboid-shaped and sphere-shaped nanofiber objects and unexpanded square and semicircular membranes. FIG. 7C provides photographs and water absorption capacity of sphere-shaped nanofiber objects with different sizes.

### DETAILED DESCRIPTION OF THE INVENTION

Accurate and rapid point-of-care tissue and microbiome sampling is critical for early detection of cancers and infectious diseases and often results in effective early intervention and prevention of disease spread. In particular, the low prevalence of Barrett's and gastric premalignancy in the Western world makes population-based endoscopic screening unfeasible and cost-ineffective. Herein, compositions and methods are provided that is useful for prescreening the general population in a minimally invasive way using a swallowable, re-expandable, ultra-absorbable, and retrievable nanofiber cuboid or sphere produced by electrospinning, gas-foaming, coating, and crosslinking. The water absorption capacity of the cuboid- and sphere-shaped nanofiber objects is shown to be ˜6000% and ˜2000% of their dry mass. In contrast, unexpanded semicircular and square nanofiber membranes showed <500% of their dry mass. Moreover, the swallowable sphere and cuboid were able to collect and release more bacteria, viruses, and cells/tissues from solutions as compared with unexpanded scaffolds. Additionally, the expanded sphere shows higher cell collection capacity from the esophagus inner wall as compared with the unexpanded nanofiber membrane. Taken together, the nanofiber capsules of the instant invention provide a minimally invasive method of collecting biological samples from the duodenal, gastric, esophagus, and oropharyngeal sites, thereby leading to timely and accurate diagnosis of many diseases. Moreover, the sample collection devices of the instant invention can also be used to deliver therapeutics (e.g., within the capsule and/or the expanded nanofiber structure) and can be used to aid in hemostasis. For example, the structures described herein may be used to treat upper gastrointestinal bleeding. The structures may also be used as a drug delivery system to specific tissues in the upper gastrointestinal tract and/or lower gastrointestinal tract (e.g., intestines and/or colon).

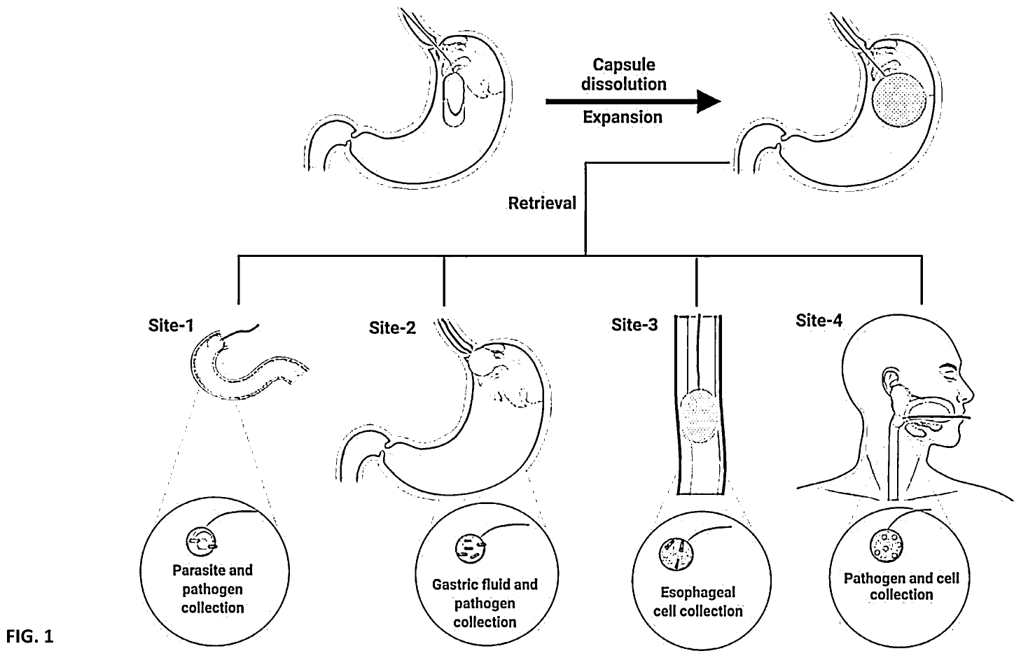

Herein, sample collection devices are provided which comprise a shape-recoverable, three-dimensional (3D) nanofiber object on a string, optionally encased in a gelatin capsule. The sample collection device is capable of obtaining samples internally or from internal organs, particularly without the need for sedation. For example, the sample collection device can be used to obtain samples from a number of locations including, without limitation: duodenal, gastric, jejunum, stomach, esophagus, oropharynx, and oropharyngeal sites (see, e.g., FIG. 1). The sample collection device could be an alternative endoscopy. The sample collection devices can be used to collect, bacteria, microbiomes, virus, fluids (e.g., gastric fluids), tissues, and/or cells (e.g., esophageal cells) from the subject. The ability to collect such samples allows for the screening of diseases and disorders such as, without limitation: Barret Esophagus, acid reflux (gastric reflux), SARS-COVID-19, Helicobacter pylori, and cancer (e.g., gastric cancer, esophageal cancer, etc.).

Briefly, 3D poly(ε-caprolactone) (PCL) nanofiber objects were fabricated by expanding two-dimensional (2D) electrospun fiber mats using an innovative gas-foaming expansion technique (Jiang, et al., ACS Biomater. Sci. Eng. (2015) 1:991-1001; Chen, et al., Appl. Phys. Rev. (2020) 7:021406; Chen, et al., Nano Lett. (2019) 19:2059-2065; Chen, et al., Adv. Mater. (2020) 32:2003754; McCarthy, et al., Nano Lett. (2021) 21:1508-1516). PCL nanofiber membranes were prepared using electrospinning. Then, the membranes were cut into either a half circular shape or a square shape in liquid nitrogen and one side of the 2D membrane was thermally fixed. The square-shaped membranes were expanded along the third dimension and half-circular-shaped membranes were expanded around the thermally fixed axis in NaBH₄ solutions to form cuboid-shaped and sphere-shaped nanofiber objects, respectively. To increase mechanical durability, the objects were coated with gelatin and crosslinked with glutaraldehyde (GA) vapor. PCL and gelatin were chosen as raw materials for making and coating nanofiber objects as they have been used in FDA-approved medical devices (Holländer, et al., J. Pharm. Sci. (2016) 105:2665-2676). In vitro sampling of microbiomes including Methicillin-resistant Staphylococcus aureus (MRSA) and SARS-CoV-2 and ex vivo sampling of cells/tissue from excised porcine esophageal walls were performed using cuboid-shaped and sphere-shaped nanofiber objects with unexpanded semicircular-shaped and square-shaped nanofiber mats as controls. The thermally welded region provided strong support to endure the elongation (pulling). The attachment between the string and the nanofiber sphere is strong enough for pulling out the sphere from the gastric site. In addition, unlike the rigid Cytosponge™, the nanofiber sphere is relatively soft and can readily accommodate the topology of the interior wall of esophagus. Considering the corners of cuboid-shaped nanofiber objects may cause discomfort of the mucosal lining, the sphere-shaped nanofiber object was used to collect cells from the lining of the esophageal wall of an excised porcine esophagus.

In accordance with the instant invention, sample collection devices (also referred to herein as nanofiber collection devices), methods of synthesizing sample collection devices, and method of using sample collection devices are provided. As described herein, the sample collection devices of the instant invention have improved collection efficiency. The sample collection devices of the instant invention can be used to collect specimen from anywhere, including any part of a subject, particularly the gastrointestinal tract (e.g., upper gastrointestinal tract).

The sample collection devices of the instant invention may be used to collect biological and/or non-biological specimens including but not limited to: fluids (e.g., gastric fluids, blood, saliva, urine, serum, plasma, etc.), exudates, bacteria, microbiomes, viruses, tissues, cells (e.g., esophageal cells) and/or subcellular materials (e.g., nucleic acid molecules, DNA, RNA, proteins, peptides, polypeptides, etc.). The sample collection devices of the instant invention may be used in medicine. The sample collection devices of the instant invention may be used, without limitation, in clinical, veterinarian, forensic, agriculture, and/or any non-clinical settings.

Generally, the sample collection devices of the instant invention comprise an expanded, nanofiber structure comprising a plurality of nanofibers. The expanded, nanofiber structure of the instant invention may be attached to a string (e.g., may be referred to as a retrieval string). The expanded, nanofiber structure will typically be at the end of the string. In certain embodiments, the expanded, nanofiber structure covers or encompasses the end of the string.

The string may be made from a variety of materials. For example, the string may comprise, without limitation: plastics and other polymers, paper and paper related materials, fibers, fabrics, and the like. In certain embodiments, the string comprises a polymer. In certain embodiments, the string comprises nylon. In certain embodiments, the string comprises polyester. In certain embodiments, the string comprises silk. In certain embodiments, the string comprises polypropylene. In certain embodiments, the string is a medical grade thread and/or yarn. Generally, the string should have a strong tensile strength such that it is not easily broken under tension as it allows for the removal of the expanded, nanofiber structure from the subject.

The nanofiber structure may be attached or fixed to the string by any means. The string may be attached to the nanofiber structure before or after expansion. In certain embodiments, the string is attached to the nanofiber structure (e.g., expanded, nanofiber structure) via an adhesive agent (e.g., epoxy, glue, cement, paste, binder, etc.). For example, the string may be attached to the thermally fused portion of the nanofiber structure or expanded, nanofiber structure, when present, via use or application of an adhesive agent. In certain embodiments, the nanofiber structure is physically and/or mechanically attached or fixed to the string. In certain embodiments, the string is thermally welded or fixed to the nanofiber structure. In certain embodiments, the string is tied or knotted to the nanofiber structure (e.g., expanded, nanofiber structure). For example, the string may be tied around the thermally fused portion of the nanofiber structure or expanded, nanofiber structure, when present.

The string can be any length. In certain embodiments, the string is less than about 150 cm in length, less than about 100 cm in length, less than about 75 cm, less than about 70 cm in length, less than about 65 cm, less than about 60 cm, less than about 55 cm, or less than about 50 cm. In certain embodiments, the string is more than about 10 cm in length, more than about 15 cm in length, more than about 20 cm in length, or more than about 25 cm in length.

The nanofiber structures of the present invention can be formed and manufactured into any shape, size, and/or thickness. For example, the nanofiber structure may have a three dimensional shape such as, without limitation: a capsule, cylinder, tube, cone, rectangle, dome, sphere (spherical), or cube (cuboidal). In certain embodiments, the nanofiber structure has a rounded geometry. For example, the nanofiber structure may be cuboidal, spherical, cylindrical, or capsular in shape. Generally, a capsular shape approximates the shape of a capsule (e.g., a geometric shape consisting of a cylinder with hemispherical ends). In certain embodiments, the expanded nanofiber structure has a spherical shape.

The nanofibers of the instant invention can be fabricated by any method. For example, the nanofiber material may be manufactured using a variety of methods including but not limited to electrospinning, phase separation, centrifugal force spinning, hypersonic spinning, and freeze-casting of short fiber solutions. In certain embodiments, the nanofiber mat or structure is synthesized by electrospinning. In certain embodiments, the expanded, nanofiber structures comprise electrospun nanofibers. The expanded nanofiber structure may comprise aligned fibers (e.g., uniaxially aligned), random fibers, and/or entangled fibers. In certain embodiments, the expanded nanofiber structure comprises aligned fibers (e.g., uniaxially, radially, vertically, or horizontally). In certain embodiments, the expanded nanofiber structure comprises radially aligned nanofibers, vertically aligned nanofiber, or a combination thereof. While the application generally describes nanofibers (fibers having a diameter less than about 1 μm (e.g., average diameter)) structures and the synthesis of three-dimensional nanofibrous structures, the instant invention also encompasses microfibers (fibers having a diameter greater than about 1 μm (e.g., average diameter)) structures and the synthesis of three-dimensional microfibrous structures.

In certain embodiments of the instant invention, the methods comprise fixing at least one point, edge, end, or side—or a portion thereof—of a nanofiber mat (sometimes referred to as 2D structure herein) and then expanding the nanofiber mat into an expanded nanofiber structure (sometimes referred to as a 3D scaffold herein). In certain embodiments, a whole or entire side of the nanofiber mat is fixed. In certain embodiments, one or more sections or portions of the nanofiber mat is fixed (e.g., the top and bottom corners on one side may be fixed). The nanofiber mat may be fixed by any means. For example, the nanofiber mat may be thermally fixed or chemically fixed. In certain embodiments, the nanofiber mat is thermally fixed.

In certain embodiments, the nanofiber mat is fixed by exposing at least one point, edge, end, or side—or a portion thereof—of the nanofiber mat to elevated temperatures (e.g., thermally fixing or thermally welding). In certain embodiments, the nanofiber mat is exposed to temperatures at or above the melting temperature of the nanofibers. In certain embodiments, the nanofiber mat is fixed by exposing at least one point, edge, end, or side—or a portion thereof—of the nanofiber mat to a temperature of at least about 50° C., 55° C., 60° C., 65° C., 70° C., 75° C., 80° C., 85° C., 90° C., 95° C., 100° C., or higher. To avoid excess fixation and/or damage to the remainder of the nanofiber mat, the exposure to elevated temperatures may be brief (e.g., less than 10 seconds, less than 5 seconds, or for about 1 second). In certain embodiments, the heat is applied perpendicularly to the nanofiber mat. In certain embodiments, the thermal fixing comprises exposing at least one point, edge, end, or side—or a portion thereof—of a nanofiber mat to about 75° C. to about 95° C., particularly about 85° C. (e.g., for less than 5 seconds, particularly about 1 second).

In certain embodiments, the nanofiber mat is chemically fixed, for example, by exposure to a chemical, solvent, or crosslinker. In certain embodiments, a chemical or solvent based method is used to fix the nanofiber mat. The chemical or solvent can be, without limitation: dichloromethane (DCM), dimethylformamide (DMF), dichloroformamide, acetone, and other organic solvents. In certain embodiments, the nanofiber mat is fixed by exposure to a crosslinker. In certain embodiments, the nanofiber mat is chemically fixed by exposing at least one point, edge, end, or side—or a portion thereof—of the nanofiber mat to a chemical, solvent, or crosslinker with minimal or no exposure the remainder of the nanofiber mat to the chemical, solvent, or crosslinker.

The methods of the instant invention may further comprise synthesizing the nanofibrous structure (e.g., mat) prior to expansion (e.g., exposure to gas bubbles). In certain embodiments, the nanofiber mat is synthesized using electrospinning. In certain embodiments, the nanofiber mat comprises aligned fibers (e.g., uniaxially), random fibers, and/or entangled fibers.

The nanofiber mat may be cut, trimmed, or shaped prior to expansion. The nanofiber mat may be cut, trimmed, or shaped prior to fixation or cut, trimmed, or shaped after fixation. In certain embodiments, the nanofiber mat is cut, trimmed, or shaped under cryogenic or frozen conditions (e.g., in liquid nitrogen). The nanofiber mat can be cut, trimmed, or shaped into any desired shape such as, without limitation: rectangles, squares, triangles, quadrangles, pentagons, hexagons, circles, ovals, semicircles, L's, C's, O's, U's, and arches. While the application generally describes nanofiber mats as the 2D structure prior to expansion, the instant invention also encompasses any nanofibrous structure which can be expanded by the methods provided herein (e.g., structures other than a mat or 3D structures which can be further expanded).

In certain embodiments, the nanofiber mat is synthesized (e.g., electrospun) as a semicircle. In certain embodiments, the nanofiber mat is cut into a semicircle. In certain embodiments, the nanofiber mat is semicircular. The nanofiber mat need not be a perfect semicircle, but rather may be semioval or semi-elliptical shapes. In certain embodiments, the length of the base of the semicircular nanofiber mat (e.g., the fixed or fused side) is less than 40 mm, less than 35 mm, less than 30 mm, less than 25 mm, less than 20 mm, less than 15 mm, or less than 10 mm. In certain embodiments, the max height of the semicircular nanofiber mat is less than 20 mm, less than 17.5 mm, less than 15 mm, less than 12.5 mm, less than 10 mm, less than 7.5 mm, or less than 5 mm.

In certain embodiments, the nanofiber mat is expanded into an expanded nanofiber structure by exposing the nanofiber mat to gas bubbles. In certain embodiments, the nanofiber mat is expanded radially and/or around a fixed axis (e.g., as defined by the fixed portion of the nanofiber mat). The bubbles can be generated by chemical reactions or physical manipulations. For example, the nanofiber mat can be submerged or immersed in a bubble/gas producing chemical reaction or physical manipulation. Generally, the longer the exposure to the bubbles, the greater the thickness and porosity of the expanded nanofiber structure increases. The nanofiber mat may also be expanded within a mold (e.g., a metal, plastic, or other material that does not expand in the presence of gas bubbles) to assist in the formation of a desired shape. The nanofiber mat may be treated with air plasma prior to exposure to gas bubbles (e.g., to increase hydrophilicity). The nanofiber mat may be exposed to gas bubbles more than once (e.g., repeatedly).

After exposure to the bubbles, the expanded nanofiber structure may be washed and/or rinsed in water and/or a desired carrier or buffer (e.g., a pharmaceutically or biologically acceptable carrier). Trapped gas bubbles may be removed by applying a vacuum to the expanded nanofiber structure. For example, the expanded nanofiber structure may be submerged or immersed in a liquid (e.g., water and/or a desired carrier or buffer) and a vacuum may be applied to rapidly remove the gas bubbles. The process may be repeated one or more times. After expansion (e.g., after rinsing and removal of trapped gas), the expanded nanofiber structure may be placed in storage in cold solution or lyophilized and/or freeze-dried.

The gas bubbles of the instant invention can be made by any method known in the art. The bubbles may be generated, for example, by chemical reactions or by physical approaches. Electrospun nanofiber mats can be expanded three dimensionally using a gas foaming based expansion method (example methods may be found in WO 2016/053988; WO 2019/060393; WO 2020/124072; and Jiang et al. (2018) Acta Biomater., 68:237-248, each incorporated herein by reference). Electrospun nanofiber mats can be expanded in the third dimension with ordered structures using gas bubbles generated by chemical reactions in an aqueous solution (see, e.g., WO 2016/053988; WO 2019/060393; Jiang et al. (2018) Acta Biomater., 68:237-248; Jiang, et al. (2015) ACS Biomater. Sci. Eng., 1:991-1001; Jiang, et al. (2016) Adv. Healthcare Mater., 5:2993-3003; Joshi, et al. (2015) Chem. Eng. J., 275:79-88; each of the foregoing incorporated by reference herein). In certain embodiments, the chemical reaction or physical manipulation does not damage or alter or does not substantially damage or alter the nanofibers (e.g., the nanofibers are inert within the chemical reaction and not chemically modified). As explained hereinabove, the nanofiber mat may be submerged or immersed in a liquid comprising the reagents of the bubble-generating chemical reaction. Examples of chemical reactions that generate bubbles include, without limitation:

NaBH₄+2H₂O═NaBO₂+4H₂

NaBH₄+4H₂O═4H₂(g)+H₃BO₃+NaOH

HCO₃⁻+H⁺═CO₂+H₂O

NH₄⁺+NO₂⁻═N₂+2H₂O

H₂CO₃=H₂O+CO₂

2H⁺+S²⁻═H₂S

2H₂O₂═O₂+2H₂O

3HNO₂=2NO+HNO₃+H₂O

HO₂CCH₂COCH₂CO₂H=2CO₂+CH₃COCH₃

2H₂O₂=2H₂+O₂

CaC₂+H₂O═C₂H₂

Zn+2HCl═H₂+ZnCl₂

2KMnO₄+16HCl=2KCl+2MnCl₂+H₂O+5Cl₂

In certain embodiments, the chemical reaction is the hydrolysis of NaBH₄ (e.g., NaBH₄+2H₂O=NaBO₂+4H₂). In certain embodiments, CO₂ gas bubbles (generated chemically or physically) are used (e.g., for hydrophilic polymers).

Examples of physical approaches for generating bubbles of the instant invention include, without limitation: 1) create high pressure (fill gas)/heat in a sealed chamber and suddenly reduce pressure; 2) dissolve gas in liquid/water in high pressure and reduce pressure to release gas bubbles; 3) use supercritical fluids (reduce pressure) like supercritical CO₂; 4) use subcritical gas liquid (then reduce pressure) (e.g., liquid CO₂, liquid propane and isobutane); 5) fluid flow; 6) apply acoustic energy or ultrasound to liquid/water; 7) apply a laser (e.g., to a liquid or water); 8) boiling; 9) reduce pressure boiling (e.g., with ethanol); and 10) apply radiation (e.g., ionizing radiation on liquid or water). The nanofiber mat may be submerged or immersed in a liquid of the bubble-generating physical manipulation.

In certain embodiments, the nanofiber mats are expanded using a subcritical or supercritical fluid or liquid (e.g., CO₂, N₂, N₂O, hydrocarbons, and fluorocarbons). In certain embodiments, the nanofiber mats are expanded by exposure to depressurized CO₂. In certain embodiments, liquid CO₂ is utilized. For example, nanofiber mats may be expanded by exposing to, contacting with or being placed into (e.g., submerged or immersed) a subcritical liquid/fluid (e.g., subcritical CO₂) and then depressurized. The cycle of placing the nanofibrous structures into subcritical CO₂ and depressurizing may be performed one or more times. Generally, the more times the expansion method is used the thickness and porosity of the nanofibrous (or microfibrous) structure increases. For examples, the cycle of exposure to subcritical CO₂ and then depressurization may be performed one, two, three, four, five, six, seven, eight, nine, ten, or more times, particularly 1-10 times, 1-5 times, or 1-3 times. In certain embodiments, the cycle of exposure to subcritical CO₂ and then depressurization is performed at least 2 times (e.g., 2-10 times, 2-5 times, 2-4 times, or 2-3 times). In certain embodiments, the method comprises placing the nanofibrous mat and dry ice (solid CO₂) in a sealed container, allowing the dry ice to turn into liquid CO₂, and then unsealing the container to allow depressurization.

The nanofiber mat and subcritical fluid (e.g., subcritical CO₂; or solid form of subcritical fluid (e.g., dry ice)) may be contained in any suitable container (e.g., one which can withstand high pressures). For example, the subcritical fluids and the nanofiber mat may be contained within, but not limited to: chambers, vessels, reactors, and tubes. In certain embodiments, the equipment or container used during the methods of the present invention will have a feature or component that allows control of the depressurization rate of the subcritical fluid. Depressurization of the subcritical fluid can be done using a variety of methods including but not limited to manually opening the container to decrease pressure or by using some type of equipment that can regulate the rate of depressurization of the reaction vessel.

The nanofibers of the instant invention may comprise any polymer. In certain embodiments, the polymer is biocompatible. In certain embodiments, the polymer is biodegradable. The polymer may by hydrophobic, hydrophilic, or amphiphilic. In certain embodiments, the polymer is hydrophobic. In certain embodiments, the polymer is hydrophilic. The polymer may be, for example, a homopolymer, random copolymer, blended polymer, copolymer, or a block copolymer. Block copolymers are most simply defined as conjugates of at least two different polymer segments or blocks. The polymer may be, for example, linear, star-like, graft, branched, dendrimer based, or hyper-branched (e.g., at least two points of branching). The polymer of the invention may have from about 2 to about 10,000, about 2 to about 1000, about 2 to about 500, about 2 to about 250, or about 2 to about 100 repeating units or monomers. The polymers of the instant invention may comprise capping termini.

Examples of hydrophobic polymers include, without limitation: poly(hydroxyethyl methacrylate), poly(N-isopropyl acrylamide), poly(lactic acid) (PLA (or PDLA)), poly(lactide-co-glycolide) (PLG), poly(lactic-co-glycolic acid) (PLGA), polyglycolide or polyglycolic acid (PGA), polycaprolactone (PCL), poly(aspartic acid), polyoxazolines (e.g., butyl, propyl, pentyl, nonyl, or phenyl poly(2-oxazolines)), polyoxypropylene, poly(glutamic acid), poly(propylene fumarate) (PPF), poly(trimethylene carbonate), polycyanoacrylate, polyurethane, polyorthoesters (POE), polyanhydride, polyester, poly(propylene oxide), poly(caprolactonefumarate), poly(1,2-butylene oxide), poly(n-butylene oxide), poly(ethyleneimine), poly(tetrahydrofurane), ethyl cellulose, polydipyrolle/dicabazole, starch, polyvinylidene fluoride (PVDF), polytetrafluoroethylene (PTFE), polydioxanone (PDO), polyether poly(urethane urea) (PEUU), cellulose acetate, polypropylene (PP), polyethylene terephthalate (PET), nylon (e.g., nylon 6), polycaprolactam, PLA/PCL, poly(3-hydroxybutyrate-co-3-hydroxyvalerate) (PHBV), PCL/calcium carbonate, and/or poly(styrene).

Examples of hydrophilic polymers include, without limitation: polyvinyl alcohol (PVA), polyvinylpyrrolidone (PVP), poly(ethylene glycol) and poly(ethylene oxide) (PEO), chitosan, collagen, chondroitin sulfate, sodium alginate, gelatin, elastin, hyaluronic acid, silk fibroin, sodium alginate/PEO, silk/PEO, silk fibroin/chitosan, hyaluronic acid/gelatin, collagen/chitosan, chondroitin sulfate/collagen, and chitosan/PEO.

Amphiphilic copolymers or polymer composites may comprise a hydrophilic polymer (e.g., segment) and a hydrophobic polymer (e.g., segment) from those listed above (e.g., gelatin/polyvinyl alcohol (PVA), PCL/collagen, chitosan/PVA, gelatin/elastin/PLGA, PDO/elastin, PHBV/collagen, PLA/hyaluronic acid, PLGA/hyaluronic acid, PCL/hyaluronic acid, PCL/collagen/hyaluronic acid, gelatin/siloxane, PLLA/MWNTs/hyaluronic acid).

Examples of polymers particularly useful for electrospinning are provided in Xie et al. (Macromol. Rapid Commun. (2008) 29:1775-1792; incorporated by reference herein; see e.g., Table 1). Examples of compounds or polymers for use in the fibers of the instant invention, particularly for electrospun nanofibers include, without limitation: natural polymers (e.g., chitosan, gelatin, collagen type I, II, and/or III, elastin, hyaluronic acid, cellulose, silk fibroin, phospholipids (Lecithin), fibrinogen, hemoglobin, fibrous calf thymus Na-DNA, virus M13 viruses), synthetic polymers (e.g., PLGA, PLA, PCL, PHBV, PDO, PGA, PLCL, PLLA-DLA, PEUU, cellulose acetate, PEG-b-PLA, EVOH, PVA, PEO, PVP), blended (e.g., PLA/PCL, gelatin/PVA, PCL/gelatin, PCL/collagen, sodium alginate/PEO, chitosan/PEO, Chitosan/PVA, gelatin/elastin/PLGA, silk/PEO, silk fibroin/chitosan, PDO/elastin, PHBV/collagen, hyaluronic acid/gelatin, collagen/chondroitin sulfate, collagen/chitosan), and composites (e.g., PDLA/HA, PCL/CaCO₃, PCL/HA, PLLA/HA, gelatin/HA, PCL/collagen/HA, collagen/HA, gelatin/siloxane, PLLA/MWNTs/HA, PLGA/HA). In certain embodiments, the nanofiber comprises polymethacrylate, poly vinyl phenol, polyvinylchloride, cellulose, polyvinyl alcohol, polyacrylamide, PLGA, collagen, polycaprolactone, polyurethanes, polyvinyl fluoride, polyamide, silk, nylon, polybennzimidazole, polycarbonate, polyacrylonitrile, polyvinyl alcohol, polylactic acid, polyethylene-co-vinyl acetate, polyethylene oxide, polyaniline, polystyrene, polyvinylcarbazole, polyethylene terephthalate, polyacrylic acid-polypyrene methanol, poly(2-hydroxyethyl methacrylate), polyether imide, polyethylene glycol, poly(ethylene-co-vinyl alcohol), polyacrylnitrile, polyvinyl pyrrolidone, polymetha-phenylene isophthalamide, gelatin, chitosan, starch, pectin, cellulose, methylcellulose, sodium polyacrylate, starch-acrylonitrile co-polymers, and/or combinations of two or more polymers. In certain embodiments, the polymer comprises polycaprolactone (PCL).

In certain embodiments, the nanofiber, nanofiber mat, and/or expanded nanofiber structure may further comprise at least one surfactant. In certain embodiments, the nanofiber, nanofiber mat, and/or expanded nanofiber structure may further comprise at least one amphiphilic block copolymer comprising hydrophilic poly(ethylene oxide) (PEO) and hydrophobic poly(propylene oxide) (PPO). In certain embodiments, the nanofiber mat and/or expanded nanofiber structure comprises a poloxamer or an amphiphilic triblock copolymer comprising a central hydrophobic PPO block flanked by two hydrophilic PEO blocks (i.e., an A-B-A triblock structure). In certain embodiments, the amphiphilic block copolymer is selected from the group consisting of Pluronic® L31, L35, F38, L42, L44, L61, L62, L63, L64, P65, F68, L72, P75, F77, L81, P84, P85, F87, F88, L92, F98, L101, P103, P104, P105, F108, L121, L122, L123, F127, 10R5, 10R8, 12R3, 17R1, 17R4, 17R8, 22R4, 25R1, 25R2, 25R4, 25R5, 25R8, 31R1, 31R2, and 31R4. In certain embodiments, the nanofiber, nanofiber mat, and/or expanded nanofiber structure comprises poloxamer 188. In certain embodiments, the nanofiber, nanofiber mat, and/or expanded nanofiber structure comprises poloxamer 407 (Pluronic® F127). The amphiphilic block copolymer (e.g., poloxamer) may be added in various amounts to the polymer solution during the synthesis process (e.g., electrospinning). In certain embodiments, about 0% to about 20%, about 0% to about 15%, about 0% to about 10%, about 0.1% to about 5%, about 0.5% to about 2%, or about 0.1% to about 1.0% (e.g., w/v) of the polymer solution is an amphiphilic block copolymer (e.g., a poloxamer (e.g., poloxamer 407)). In certain embodiments, about 0.1% to about 50%, about 0.1% to about 40%, about 0.1% to about 30%, about 0.1% to about 25%, about 0.1% to about 20% (e.g., w/v) of the polymer solution is polymer (e.g., PCL).

In certain embodiments, the polymer solution comprises about 10% polymer (w/v) (e.g., PCL) and about 1.0% poloxamer 407 (w/v) (Pluronic® F127). In certain embodiments, the polymer solution comprises PCL and poloxamer 407 in a ratio (e.g., by weight) of about 200:1 to about 2:1, about 100:1 to about 2:1, about 50:1 to about 4:1, about 20:1 to about 5:1, about 15:1 to about 7.55:1, or about 10:1.

In certain embodiments, the nanofibers and/or nanofiber structures are coated with additional materials to enhance their properties. In certain embodiments, the expanded nanofiber structure, optionally attached to the string, is coated with the additional material. For example, the nanofibers and/or nanofiber structure can be coated with a material to help reinforce the structure and increase fluid absorption. In certain embodiments, the nanofibers and/or nanofiber structure may be coated with proteins, collagen, fibronectin, collagen, a proteoglycan, elastin, or a glycosaminoglycans (e.g., hyaluronic acid, heparin, chondroitin sulfate, or keratan sulfate). In certain embodiments, the nanofibers and/or nanofiber structures comprise a material that enhances the nanofiber structure's ability to absorb fluids, particularly aqueous solutions (e.g., blood), and/or allow for the 3D shapes/structures of the expanded nanofiber structure to be recoverable after compression. In certain embodiments, the nanofibers comprise a polymer and the material which enhances the absorption properties.

In certain embodiments, the nanofibers and/or nanofiber structures are coated with the material which enhances the absorption properties. The term “coat” refers to a layer of a substance/material on the surface of a structure. Coatings may, but need not, also impregnate the nanofiber structure. Further, while a coating may cover 100% of the nanofibers and/or nanofiber structure, a coating may also cover less than 100% of the surface of the nanofibers and/or nanofiber structure (e.g., at least about 75%, at least about 80%, at least about 85%, at least about 90%, at least about 95%, at least about 98%, or more the surface may be coated). Materials which enhance the absorption properties of the expanded nanofiber structures include, without limitation: gelatin, gelatin methacryloyl (GelMA), alginate, chitosan, collagen, starch, pectin, cellulose, methylcellulose, sodium polyacrylate, starch-acrylonitrile co-polymers, other natural or synthetic hydrogels, and derivatives thereof (e.g., del Valle et al., Gels (2017) 3:27). In certain embodiments, the material is a hydrogel (e.g., a polymer matrix able to retain water, particularly large amounts of water, in a swollen state). In certain embodiments, the material is gelatin, gelatin methacryloyl (GelMA), and/or chitosan. In certain embodiments, the material is gelatin. In certain embodiments, the expanded nanofiber structure is coated with the coating material (e.g., gelatin) by submersion into a solution of the coating material (e.g., gelatin). In certain embodiments, the expanded nanofiber structures are coated with about 0.05% to about 20%, about 0.1% to about 20%, about 0.1% to about 10%, or about 0.1% to about 1% coating material (e.g., gelatin) (e.g., by weight or w/v). In certain embodiments, the coating material (e.g., hydrogel) is freeze-dried after coating. In certain embodiments, the coating material (e.g., hydrogel) is crosslinked (e.g., by glutaraldehyde or genipin) after coating (e.g., after freeze-drying).

In certain embodiments, the nanofiber structures of the instant invention are crosslinked (e.g., before or after expansion). In certain embodiments, the expanded nanofiber structure is crosslinked after coating. Crosslinking may be done using a variety of techniques including thermal crosslinking, chemical crosslinking, UV-crosslinking, and photo-crosslinking. For example, the nanofiber structures of the instant invention may be crosslinked with a crosslinker such as, without limitation: formaldehyde, paraformaldehyde, acetaldehyde, glutaraldehyde, a photocrosslinker, genipin, and natural phenolic compounds (Mazaki, et al., Sci. Rep. (2014) 4:4457; Bigi, et al., Biomaterials (2002) 23:4827-4832; Zhang, et al., Biomacromolecules (2010) 11:1125-1132; incorporated herein by reference). In certain embodiments, the crosslinker is glutaraldehyde. In certain embodiments, the crosslinker is genipin. The crosslinker may be a bifunctional, trifunctional, or multifunctional crosslinking reagent. In certain embodiments, the crosslinker is glutaraldehyde. In certain embodiments (e.g., instead of crosslinking or in addition to crosslinking), the nanofiber structure is thermally treated (e.g., at a temperature close to, but below, the melting point of the nanofibers; e.g., about 50° C.).

In certain embodiments, the nanofiber structures are loaded with and/or coated with an active agent such as a drug, biologic molecule, cell based therapy, or tissue based therapy. In a particular embodiment, the expanded, nanofibrous structure comprises or encapsulates at least one agent (e.g., a therapeutic agent, growth factor, signaling molecule, cytokine, hemostatic agent, antibiotic, a cell type (e.g. stem cells), etc.). In a particular embodiment, the coating comprises or encapsulates at least one agent (e.g., a therapeutic agent, growth factor, signaling molecule, cytokine, hemostatic agent, antibiotic, a cell type (e.g. stem cells), etc.).

In certain embodiments, the nanofiber structures of the instant invention are inserted into a capsule, particularly a swallowable capsule. Generally, the string attached to the nanofiber structure is accessible outside the capsule and/or remains largely outside the capsule. In certain embodiments, the capsule is a rapid release capsule, particularly in the low release environment of the stomach. In certain embodiments, the capsule comprises gelatin. In certain embodiments, the capsule is size 000, 00, 0, 1, 2, 3, or 4.

The expanded nanofiber structures and/or sample collection devices of the instant invention may also be sterilized. For example, the expanded nanofiber structures can be sterilized using various methods (e.g., by treating with ethylene oxide gas, gamma irradiation, or 70% ethanol). In certain embodiments, the expanded nanofiber structure and/or sample collection device are sterilized by treating with ethylene oxide.

The instant application also encompasses the sample collection devices synthesized by the methods of the instant invention. Compositions comprising the sample collection devices synthesized by the methods of the instant invention and at least one pharmaceutically or biologically acceptable carrier are also encompassed by the instant invention.

In accordance with the instant invention, sample collection devices are provided. In certain embodiments, the sample collection device comprises an expanded, nanofiber structure and a string, wherein the expanded, nanofiber structure is attached to the string (e.g., at one end of the string). In certain embodiments, the expanded, nanofiber structure is as described herein. In certain embodiments, the string is as described herein.

In accordance with the instant invention, methods of collecting a sample (e.g., a biological sample) from a subject are provided. In certain embodiments, the method comprises contacting a sample collection device of the instant invention with a site in the subject. In certain embodiments, the method further comprises removing the expanded, nanofiber structure from the subject by using and/or pulling the retrieval string. In certain embodiments, the site is along the gastrointestinal tract of the subject, particularly the upper gastrointestinal tract. In certain embodiments, the site is duodenal, gastric, esophagus, and/or oropharyngeal site. In certain embodiments, the methods comprises the subject swallowing the sample collection device. The site may be contacted more than one time with the sample collection devices of the instant invention. The sample is collected from the site in the subject via the expanded nanofiber structure of the sample collection devices. In certain embodiments, the sample comprises a cell. In certain embodiments, the sample comprises a tissue. In certain embodiments, the sample comprises a fluid. In certain embodiments, the sample comprises bacteria. In certain embodiments, the sample comprises a virus (e.g., a SARS-COV-2 virus). In certain embodiments, the sample comprises a nucleic acid molecule (e.g., DNA, RNA, mRNA, etc.). In certain embodiments, the sample comprises a polypeptide, protein, or peptide. In certain embodiments, the site is within the subject's head (e.g., into a cavity (e.g., nasal cavity, nasopharynx cavity, or oropharynx). In certain embodiments, the site is contacted more than one time with the sample collection devices. In certain embodiments, the method further comprises placing the expanded nanofiber structure in a container (e.g., a tube). In certain embodiments, the container comprises a carrier. In certain embodiments, the carrier preserves the sample dislodged from the expanded nanofiber structure. In certain embodiments, the sample obtained from the subject is analyzed (e.g., identified). For example, the sample can be analyzed for the presence of Barret's esophagus, bacterial infection, viral infection, cancer (e.g., gastric cancer, esophageal cancer, etc.), and the like.

### Definitions

The singular forms “a,” “an,” and “the” include plural referents unless the context clearly dictates otherwise.

As used herein, the term “electrospinning” refers to the production of fibers (i.e., electrospun fibers), particularly micro- or nano-sized fibers, from a solution or melt using interactions between fluid dynamics and charged surfaces (e.g., by streaming a solution or melt through an orifice in response to an electric field). Forms of electrospun nanofibers include, without limitation, branched nanofibers, tubes, ribbons and split nanofibers, nanofiber yarns, surface-coated nanofibers (e.g., with carbon, metals, etc.), nanofibers produced in a vacuum, and the like. The production of electrospun fibers is described, for example, in Gibson et al. (1999) AlChE J., 45:190-195.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., TrisHCl, acetate, phosphate), water, aqueous solutions, oils, bulking substance (e.g., lactose, mannitol), excipient, auxiliary agent or vehicle with which an active agent of the present invention is administered. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients (3rd Ed.), American Pharmaceutical Association, Washington.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

“Hydrophobic” designates a preference for apolar environments (e.g., a hydrophobic substance or moiety is more readily dissolved in or wetted by non-polar solvents, such as hydrocarbons, than by water). In certain embodiments, hydrophobic polymers may have aqueous solubility less than about 1% wt. at 37° C. In certain embodiments, polymers that at 1% solution in bi-distilled water have a cloud point below about 37° C., particularly below about 34° C., may be considered hydrophobic.

As used herein, the term “hydrophilic” means the ability to dissolve in water. In a particular embodiment, polymers that at 1% solution in bi-distilled water have a cloud point above about 37° C., particularly above about 40° C., may be considered hydrophilic.

As used herein, the term “amphiphilic” means the ability to dissolve in both water and lipids/apolar environments. Typically, an amphiphilic compound comprises a hydrophilic portion and a hydrophobic portion.

As used herein, the term “subject” refers to an animal, particularly a mammal, particularly a human.

The term “hydrogel” refers to a water-swellable, insoluble polymeric matrix (e.g., hydrophilic polymers) comprising a network of macromolecules, optionally crosslinked, that can absorb water to form a gel.

The term “crosslink” refers to a bond or chain of atoms attached between and linking two different molecules (e.g., polymer chains). The term “crosslinker” refers to a molecule capable of forming a covalent linkage between compounds. A “photocrosslinker” refers to a molecule capable of forming a covalent linkage between compounds after photoinduction (e.g., exposure to electromagnetic radiation in the visible and near-visible range). Crosslinkers are well known in the art (e.g., formaldehyde, paraformaldehyde, acetaldehyde, glutaraldehyde, etc.). The crosslinker may be a bifunctional, trifunctional, or multifunctional crosslinking reagent.

The following example illustrates certain embodiments of the invention. It is not intended to limit the invention in any way.

### EXAMPLE

### Materials and Methods

### Materials

PCL (Mw=80 kDa), gelatin (porcine), Pluronic® F-127, and NaBH₄ were all purchased from Sigma-Aldrich (St. Louis, MO). Dichloromethane (DCM) and N, N-dimethylformamide (DMF) were purchased from Oakwood Chemical (Estill, SC). Black polyester strings were purchased locally in Omaha (NE). MRSA USA300 LAC was obtained in-house at the University of Nebraska Medical Center (UNMC). Tryptic soy broth (TSB) bacterial medium purchased from Thermo Fisher Scientific In. (Waltham, MA). SARS-COV-2 (Strain: BEI_USA-WA1/2020) was obtained from BEI Resources. Phosphate Buffered Saline (PBS) was purchased from Fisher Scientific (Hampton, NH). QIAamp® Viral RNA Mini Kit was purchased from Qiagen (Venlo, Netherlands). RT-PCR (2X) Master Mix and SuperScript™ III Platinum Taq Mix were purchased from Invitrogen (Carlsbad, CA). Primers and Probe for RT-PCR were purchased from Integrated DNA Technologies (Coralville, IA). QuantStudio™ 3 instrument and software purchased from Applied Biosystems (Foster City, CA).

### Fabrication of PCL Nanofiber Mats

PCL nanofiber mats were generated via electrospinning (Jiang, et al., ACS Biomater. Sci. Eng. (2015) 1:991-1001; Chen, et al., Appl. Phys. Rev. (2020) 7:021406; Chen, et al., Nano Lett. (2019) 19:2059-2065; Chen, et al., Adv. Mater. (2020) 32:2003754; McCarthy, et al., Nano Lett. (2021) 21:1508-1516). PCL pellets (10% w/v) and Pluronic® F-127 (1.0% w/v) were dissolved in a 4:1 (v/v) DCM: DMF solution. The resulting PCL/F-127 solution was electrospun in 16 ml/hour at room temperature under applied voltage 15 kV using a LE-100 electrospinning machine with a circular 20 emitter array equipped with 21-gage needles (such that each needle had a flow rate of 0.8 ml/hour). The nanofibers were collected on a high-speed drum collector until an approximately 1 mm thick mat was obtained.

### Fabrication of Expanded Cuboid- and Sphere-Shaped Nanofiber Objects

The fabrication of expanded cuboid- and sphere-shaped nanofiber objects were performed (Jiang, et al., ACS Biomater. Sci. Eng. (2015) 1:991-1001; Chen, et al., Adv. Mater. (2020) 32:2003754). Specifically, the nanofiber mat was removed from the drum and cut into different sized squares while submerged in liquid nitrogen, including a rectangle (10 mm×10 mm) and a semicircle (diameter 8, 10, 15, and 20 mm). The mats were completely soaked in liquid nitrogen before cutting to ensure that no fusion occurred between the fibers. The nanofiber square and semicircle mats were removed from the liquid nitrogen and thermally fused (only the semicircle) along with one of the long sides (applying heat perpendicular to the face of the semicircle) by melting the edge on an 85° C. hot plate. Once the semicircles were cooled, they were placed into a 1 M NaBH₄ solution and left for 10-30 minutes until they expanded circularly. After expansion, the cuboid- and sphere-shaped nanofiber objects were washed with distilled water three times and expanded under a vacuum at 200 Pa three times for 10-15 seconds. The initially expanded objects without gelatin coating/crosslinking would shrink to some degree when washed/immersed in water. After washing, vacuum was used to assist the expansion as the vacuum causes bubbles growth rapidly, leading to the expansion. After the third vacuum, all the water was removed, and the expanded objects were freeze-dried. Once dry, the nanofiber objects were removed and submerged in a 0.5% gelatin solution and allowed to soak for 20 minutes. Gelatin-coated nanofiber objects were directly freeze-dried again. Once dry, the nanofiber objects were crosslinked in a glutaraldehyde (GA) chamber for 24 hours. The glutaraldehyde chamber consisted of a closed system (air-tight polyethylene terephthalate box (76.2 cm×38.1 cm×38.1 cm)) with 2 custom fans to circulate the GA vapor. One ml GA in ethanol (25%) in a petri dish was added to the bottom of the GA chamber, from which the vapor arose from. The fans were continually running throughout the duration of crosslinking to ensure sufficient penetration of the GA vapor into the nanofiber samples. During the 24-hour crosslinking, the ethanol containing GA circulated in the chamber and crosslinked with gelatin via Schiff base chemistry.

### Characterization of Expanded Nanofiber Objects

Morphology analysis of the expanded nanofiber objects (cuboid and sphere) was performed to understand the nanofibrous microstructure even after the expansion. In this study, each expanded nanofiber object like cuboid and sphere was imaged using scanning electron microscopy (SEM) (FEI, Quanta 200, Oregon) (Jiang, et al., ACS Biomater. Sci. Eng. (2015) 1:991-1001; Chen, et al., Adv. Mater. (2020) 32:2003754). The nanofiber objects (cuboid and sphere) were mounted onto a metallic stub using double-sided conductive carbon tape and then sputter-coated in the Ar atmosphere with an Au—Pd target at a peak current of 15 μA for 5 minutes. The expanded nanofiber objects were subsequently imaged using an accelerating voltage of 15-25 kV.

### Fabrication of the Nanofiber Capsule on a String

The cuboid- and sphere-shaped nanofiber objects were tied with polyester strings. Briefly, a polyester string was used to make a knot in a thermally welded part of the nanofiber sphere and semicircular mats. For the cuboid-shaped nanofiber object and square membrane, one side was compressed and tied with a polyester string. Then, the tied cuboid- and sphere-shaped nanofiber objects and semicircular and square mats were encased into the empty gelatin capsules. Furthermore, according to the size of objects, different sizes of the capsules can be made, for example, Size 000:800-1600 mg, Size 00:600-1100 mg, Size 0:400-800 mg, Size 1:300-600 mg, Size 2:200-400 mg, Size 3:150-300 mg, Size 4:120-240 mg. In this study, 4 different diameters were prepared changing from 8 mm to 20 mm which were encapsulated in different empty gelatin capsules. Finally, each capsule was sterilized with ethylene oxide (ETO) prior to use.

### Monitoring the Capsule Dissolution and Re-Expansion of Nanofiber Objects

In this study, different nanofiber objects were encased into the empty gelatin capsules (Size 0). Then, each sample was dipped into the different pH solutions like pH, 2.0, 4.0, 6.0, and 7.4 (n=3) and the capsules dissolution and re-expansion of objects were monitored using a timer.

### Tensile Strength Testing

To ascertain that the nanofiber capsule on a string has sufficient mechanical strength to withstand any supraphysiological resistance during retrieval, the tensile testing to failure was performed. Each group (n=5) (cuboid- and sphere-shaped nanofiber objects and semicircular and square nanofiber mats) was subject to an ultimate tensile test using a CellScale UniVert (CellScale biomaterials testing, Waterloo, ON, CA) with a 200 N load cell. Each nanofiber capsule on a string was gripped under the string connection point as to not interfere with the mechanical strength of the string attachment site. The string ends (which would be pulled on during removal) were wrapped around and clamped within the clamp grip of the moving head of the machine. During fixation, the string was slack. After removing the string slack by adjusting the vertical position of the testing device, a tensile test at a fixed speed of 10 mm/second was conducted until failure was achieved. Failure included string pull-out from the sample and string break. Displacement data was displayed as the median sample and ultimate tensile strength (tensile break force) was computed from the 5 replicates of each sample. Data were displayed as the mean±standard deviation from the maximum force prior to mechanical failure.

Tissue Swabbing from Porcine Esophagus Explants

The tissue collection efficiency of nanofiber spheres was performed using pig esophagus explants. The pig esophagus was collected from the euthanized pigs from the animal facility at the University of Nebraska Medical Center with an IACUC-approved animal protocol 19-053-06-FC. The nanofiber spheres and semicircular membranes were passed through the 10-15 cm pig esophagus and the diameter was 20 mm. The maximum extraction forces were measured by a spring balance. The measurement was repeated three times. Then, H&E staining was performed to identify the cells on the nanofiber spheres and semicircular membranes.

### Bacterial Swabbing

The bacterial specimen collection efficiency of the nanofiber capsules was investigated. Single bacterial colonies of MRSA were picked up by inoculating loops and cultured at 37° C. and 200 rpm in liquid TSB overnight. Ten microliters of bacterial culture were added into 2 ml of fresh TSB and incubated for an additional 2 hours. Then, the cultures were centrifuged and washed with PBS twice. Bacteria were resuspended and then diluted into different dilutions of MRSA from 10³ to 10⁶ CFU/ml. Then, the sterilized nanofiber capsules were dipped for 10 seconds and the specimen were collected from different dilutions and make up with 2 ml sterilized PBS. Then the 50 μl of the solution were inoculated on agar plates and spread with an L-shaped spreader. The plate was then incubated in an incubator for 12 hours at 37° C. The CFU numbers of bacteria were determined by counting the number of colonies from the plate.

### SARS-COV-2 Swabbing and Detection

The method for SARS-COV-2 swabbing and detection was similar to that described (McCarthy, et al., Nano Lett. (2021) 21:1508-1516). Briefly, an initial stock of SARS-COV-2 with a titer of 1.30×10⁵ plaque-forming units per ml (pfu/ml) was serially diluted in PBS by single log dilution down to 1.30×10° pfu/ml. A copy of each dilution was created for each nanofiber objects (cuboid- and sphere-shaped nanofiber objects and square and semicircular membranes) in 50 ml conical tubes for a total of 3 tubes per dilution. Each nanofiber object was dipped into its dilution tube and pressed against the insides of its respective 1.5 ml microcentrifuge tube already containing 500 μl of PBS for sample collection. Each 1.5 ml microcentrifuge tube was vortexed for 10 seconds to ensure a thorough sample mixture into the PBS. The sample collection was conducted in triplicates for each swab type and dilution. Negative control for each swab type consisted of the 50 ml conical tube containing PBS only while repeating the same sample collection method. 140 μl of each collected sample was taken for RNA isolation via Qiagen QIAamp™ Viral RNA Mini Kit. The viral RNA was analyzed by RT-PCR. The RT-PCR was performed on the QuantStudio™ 3. The RT-PCR reactions underwent an initial condition of 55° C. for 10 minutes then a 94° C. for 4 minutes followed by 45 cycles of 94° C. for 15 seconds and 58° C. for 30 seconds. The RT-PCR blank sample control resulted in no amplification. Each RT-PCR reaction consisted of: 5.6 μl nuclease-free water, 12.5 μl Invitrogen (2×) Master Mix, 0.4 μl MgSO₄, μl Primer/Probe Mix* [0.15 μl Forward Primer (100 μM stock), 0.2 μl Reverse Primer (100 μM stock), 0.05 μl Probe (100 μM stock), 0.6 μl TE Buffer], 0.5 μl SuperScript™ III Platinum Taq Mix, 5.0 μl extracted Sample RNA for a 25.0 μl Total Volume. *E gene target primers and probe:

Probe:
(SEQ ID NO: 1)
5′/56-FAM/ACACTAAGCCATCCTTACTGCGCTTCG/3AIBkFG/-3′
Forward Primer:
(SEQ ID NO: 2)
5′-ATATTGCAGCAGTACGCACACA-3′
Reverse Primer:
(SEQ ID NO: 3)
5′-ACAGGTACGTTAATAGTTAATAGCGT-3′

### Statistics

All experiments and measurements were done with a minimum of three replicates. Data are expressed in tables and graphs as the mean±standard deviation. All physical measurements were taken on a calibrated digital caliper, and all photographic measurements were made using ImageJ. Ordinary one-way ANOVAs, paired and unpaired t-tests, two-way ANOVAs (displaying significance within-group), and frequency distributions were used where appropriate. Post-hoc testing (Tukey's), residual distribution, and normality was checked following each analysis. Definitive outliers were identified and removed using Iterative Grubbs' testing. Interpretation of the SARS-CoV-2 data included within-group comparisons except at 101 and 10° pfu/ml, where no virus was detected. Each data set was analyzed and charted using GraphPad Prism Version 9.0.0. Significance was denoted as ns≥0.05, 0.01<*p<0.05, 0.01<**p<0.001, 0.001<***p<0.0001, ****p<0.0001.

### Results

### Preparation and Characterization of Expandable Nanofiber Capsule on a String

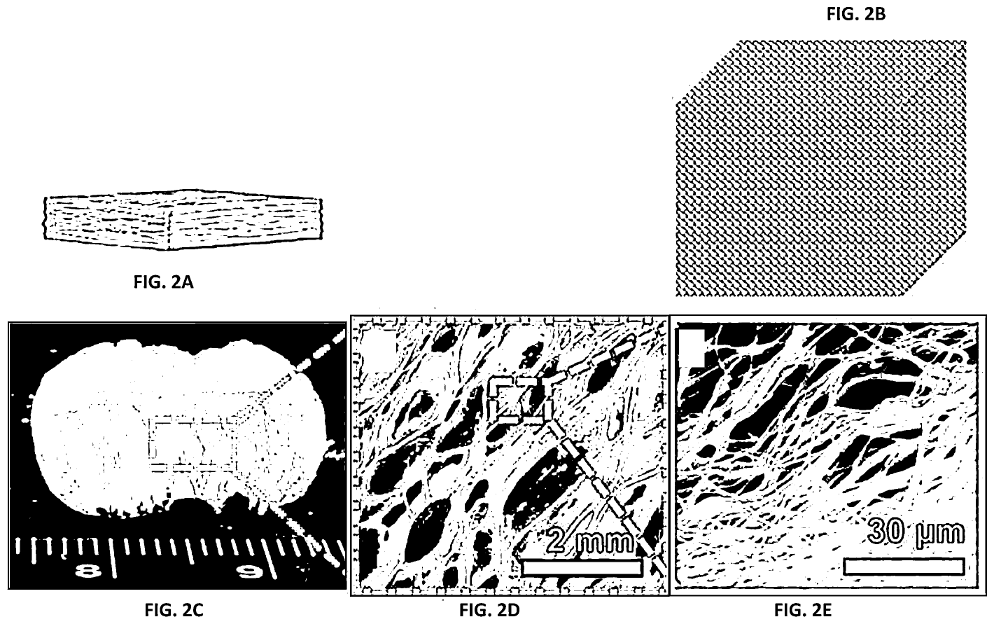

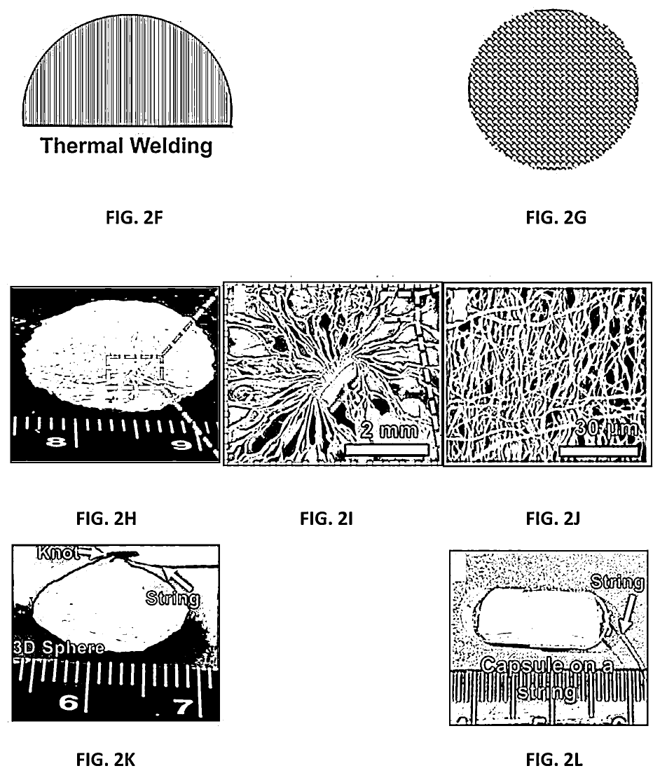

FIGS. 2A and 2B show a schematic illustrating the expansion of a 2D nanofiber membrane into a cuboid-shaped nanofiber object through an innovative gas-foaming technology (Jiang, et al., ACS Biomater. Sci. Eng. (2015) 1:991-1001; Chen, et al., Appl. Phys. Rev. (2020) 7:021406; Chen, et al., Nano Lett. (2019) 19:2059-2065; Chen, et al., Adv. Mater. (2020) 32:2003754; McCarthy, et al., Nano Lett. (2021) 21:1508-1516). The expanded objects were coated with 0.5% gelatin and crosslinked with GA. FIG. 2C shows a photograph of a cuboid-shaped nanofiber object. FIGS. 2D and 2E show the morphology of cuboid-shaped nanofiber objects, indicating the porous and fibrous structure. Similarly, FIGS. 2F and 2G show a schematic illustrating the transformation of a semicircular membrane into a nanofiber sphere via a solids-of-revolution-inspired gas-foaming expansion (Chen, et al., Appl. Phys. Rev. (2020) 7:021406; Chen, et al., Nano Lett. (2019) 19:2059-2065; Chen, et al., Adv. Mater. (2020) 32:2003754). FIG. 2H shows a photograph of a nanofiber sphere. FIGS. 2I and 2J show SEM images of the nanofiber sphere, indicating the radial pattern and fiber alignment. Here, the detachment by thermally welded common tethering point in the string was overcome (FIG. 2I, marked as a rectangle). FIG. 2K shows a photograph of a nanofiber sphere on a string after coating with 0.5% gelatin and crosslinking with GA via Schiff base chemistry provided mechanical stability and re-expansion capacity of the nanofiber objects. The excessive GA vapors were quenched by Tris Buffer (pH 8.0). Alternatively, GA can be replaced with genipin—a natural crosslinking low-toxic agent. Such a nanofiber sphere on a string can be encapsulated in a swallowable capsule through compression and insertion as shown in FIG. 2L.

### Water Absorption Capacity of Expanded Nanofiber Objects

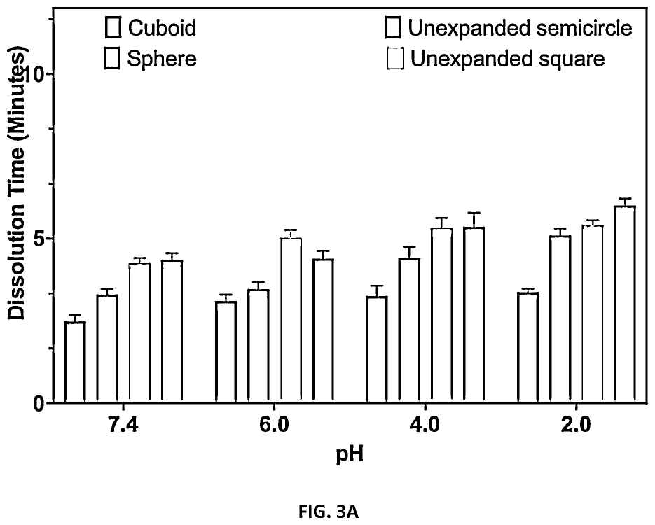

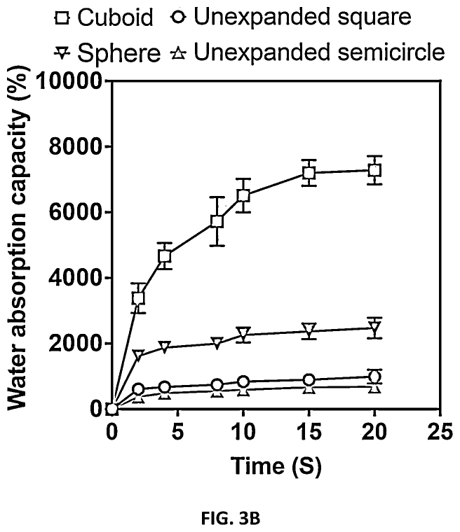

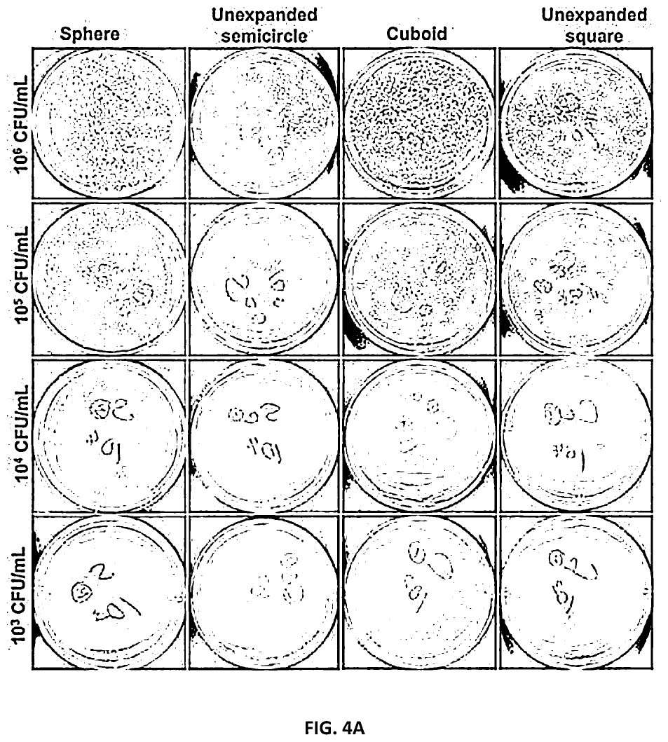

To demonstrate the device's ability to collect liquid specimens from gastric and duodenal sites, the water absorption capacity of expanded, cuboid- and sphere-shaped nanofiber objects was examined. The unexpanded counterparts were used for comparison. FIG. 3B shows the water absorption capacity of expanded nanofiber objects and unexpanded counterparts. Cuboid- and sphere-shaped nanofiber objects absorbed ˜6000% and ˜2000% of their dry mass. In contrast, unexpanded semicircular and square nanofiber membranes absorbed <500% of their dry mass. The unexpanded 2D nanofiber membranes showed less absorption due to their densely packed structures (Chen, et al., Biomaterials (2018) 179:46). The expanded, cuboid-shaped nanofiber objects showed the highest water absorption capacity probably because of their higher porosity when compared with spherical nanofiber objects.

### Shell Dissolution Capacity of Compressed Nanofiber Object in the Empty Gelatin Capsule

To simulate the sampling of saliva and gastric sites, the dissolution time of gelatin capsules after encapsulation with 3D nanofiber objects and 2D nanofiber membranes was investigated at different pHs including 2.0, 4.0, 6.0, and 7.4. The dissolution process of capsules encased with compressed cuboid-shaped nanofiber objects was determined. The gelatin capsules with compressed cuboid-shaped nanofiber objects were broken after 145 seconds (2.3 minutes) at pH 7.4 and 190 seconds (3.1 minutes) at pH 2.0. The capsule shell was completely dissolved after 190 seconds (3.1 minutes) at pH=2.0 and then the compressed cuboid-shaped nanofiber object was re-expanded and recovered its shape as anticipated. The dissolution process of capsules encapsulated with compressed spherical nanofiber objects was determined. The gelatin capsules were broken after 190 seconds (3.1 minute) at pH 7.4 and 285 seconds (4.95 minute) at pH 2.0. Similarly, the capsule shell was totally disintegrated, and the compressed nanofiber sphere returned to its original shape after 285 seconds (4.3 minute) at pH=2.0. The dissolution process of capsules loaded with unexpanded nanofiber square and semicircular membranes was also determined. Capsules loaded with unexpanded, square, and semicircular nanofiber mats had dissolution times of 252 seconds (4.2 minutes) and 324 seconds (5.4 minutes) at pHs of 7.4 and 2, respectively. The disintegration rate of capsules loaded with unexpanded nanofiber objects (square and semicircular membranes) was slower when compared with the ones encapsulated with compressed cuboid- and sphere-shaped nanofiber objects (FIG. 3A). This could be because of their ultra-absorption capability and re-expandable property. In addition, in all the cases, the shell of capsules dissolved faster in the solutions with higher pHs.

### Sampling of Bacterial and Viral Specimens

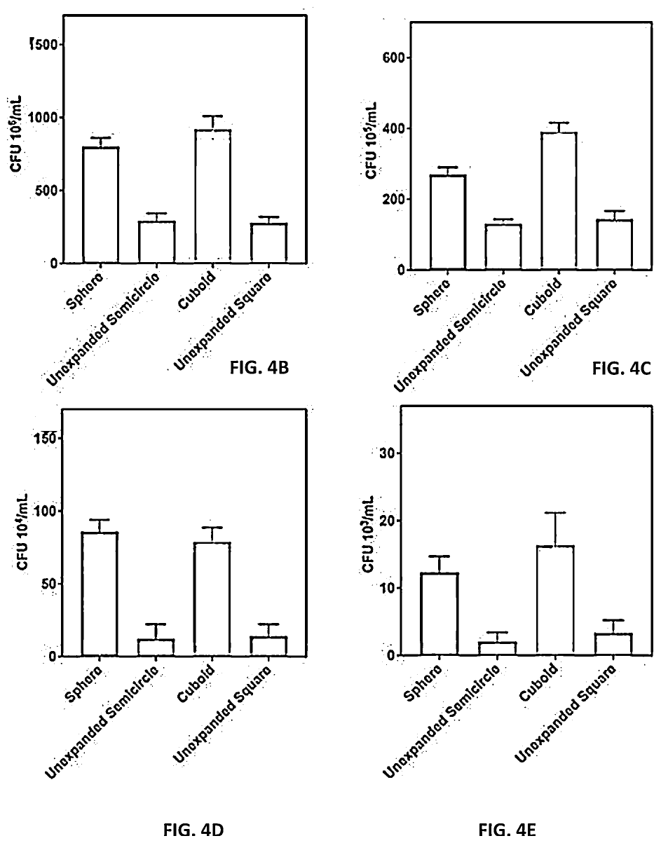

Sampling pathogens is one of the significant steps in disease diagnosis (Patel, et al., World J. Gastroenterol. (2014) 20:12847; Gastli, et al., J. Clin. Med (2021) 10:2755). When detecting bacteria or viruses, maintaining high sensitivity is critical for avoiding false-negative test rates, especially in patients with low titers or bacterial counts (Chu, et al., N. Engl. J. Med (2020) 383:185-187; Ashford, et al., Emerg. Infect. Dis. (2003) 9:515-519). To demonstrate pathogen collecting capabilities, methicillin-resistant Staphylococcus aureus (MRSA) was sampled as a model bacterium from a series of diluted solutions using unexpanded nanofiber membranes and expanded 3D nanofiber objects. After sampling, nanofiber membranes and objects were used to inoculate an L.B. agar plate through a spreading method (FIG. 4A), and colonies were counted after incubation overnight (FIGS. 4B-4E). Expanded, cuboid and sphere-shaped nanofiber objects were able to collect 10 times more bacteria from solutions with the same bacterial concentration due to the inner porous structure, and expansion capability. The sampling from the high bacterial concentration 10⁶ CFU/ml (FIG. 4B), the sphere and cuboid showed significantly higher sensitivity compared to cotton swabs. The sphere and cuboid achieved almost 10-12 times higher colony counts as compared with unexpanded nanofiber objects. FIG. 4B shows that 800±60 and 920±89 bacterial colonies were counted from the sphere and cuboid when sampling from 10⁶ CFU/ml bacterial solutions as compared with unexpanded nanofiber objects 291±52 and 280±39. These results were consistent even in low bacterial concentrations 10³ CFU/ml (FIG. 4E). FIG. 4E shows that 12.33±2 and 16±5 bacterial colonies were counted from the sphere and cuboid when sampling from 10³ CFU/ml bacterial solutions as compared with unexpanded nanofiber objects 2±1.4 and 3±1.5. In general, bacteria-containing droplets are absorbed onto the swab and detected by RT-PCR, morphological phenotyping, or quadrant streaking and culture. Due to their ultrahigh absorptivity and release of biological specimens, the 3D nanofiber objects may be used for many routine postharvest analyses.

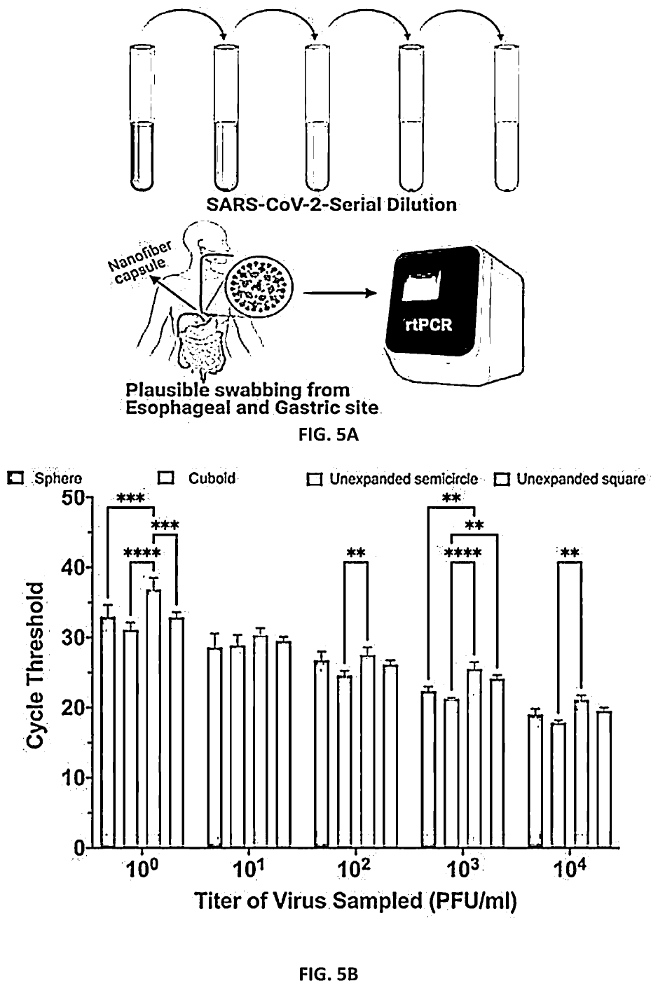

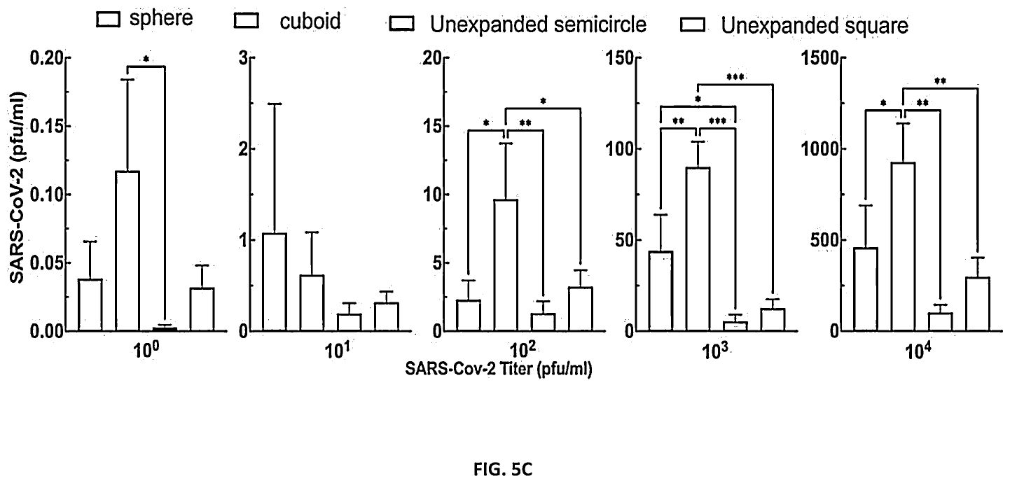

SARS-COV-2 viruses are commonly swabbed through the nose, mouth, or throat (Patel, et al., World J. Gastroenterol. (2014) 20:12847; Gastli, et al., J. Clin. Med (2021) 10:2755). To diagnose the virus at the early stage, sensitive swabs are needed to collect as many viral particles as possible (Watts, G., Lancet (2018) 391:2593-2594; Etzioni, et al., Nat. Rev. Cancer (2003) 3:243-252; Chu, et al., N. Engl. J. Med. (2020) 383:185-187; Ashford, et al., Emerg. Infect. Dis. (2003) 9:515-519; Patel, et al., World J. Gastroenterol. (2014) 20:12847; Gastli, et al., J. Clin. Med (2021) 10:2755). Increased sampling sensitivity can result in a decrease in false-negative tests, which is vital for early intervention and diagnosis. Generally, nasopharyngeal and throat sites are preferred locations for collecting SARS-COV-2 specimens for diagnostic testing, with both showing equivalent sensitivities (Martinez, R. M., Clin. Microbiol. Newsl. (2020) 42:121; Valentine-Graves, et al., medRxiv (2020) 12:2020.06.10.20127845). Due to shortages of standard collection kits and varying regional availability of swabs and collection containers at patient testing locations, other acceptable specimen types for SARS-COV-2 and other respiratory virus testing may include nasopharyngeal aspirate, endotracheal aspirate, bronchoalveolar lavage (BAL), and bronchial washes (Cipriano, et al., Cureus (2020) 12:7422; Guiney, et al., Br. J. Clin. Pharmacol. (2011) 72:133-142). However, researchers are still learning about new symptoms that present in different body parts, including the stomach and esophagus; therefore, collecting a specimen from the esophagus and stomach could be an option for diagnosis. In this study, the viral collecting efficiency of 3D cuboid- and sphere-shaped nanofiber objects was compared to nanofiber membranes by swabbing a serial dilution of SARS-COV-2 (FIG. 5A). After swabbing, nanofiber materials were squeezed and vortexed in a buffer and PCR amplification of SARS-COV-2 was carried out. At 10°-10⁴ pfu/ml, each type of nanofiber materials can identify SARS-COV-2, but cuboid- and sphere-shaped nanofiber objects required fewer PCR cycles at most titers compared to 2D nanofiber mats (FIG. 5B). At titers of 10°, 10², 10³, and 10⁴, cuboid-shaped nanofiber objects were able to collect more viruses when compared with unexpanded semicircular nanofiber mats (FIG. 5C).

Minimally Invasive Sample Collection from Swine Esophagus Explants

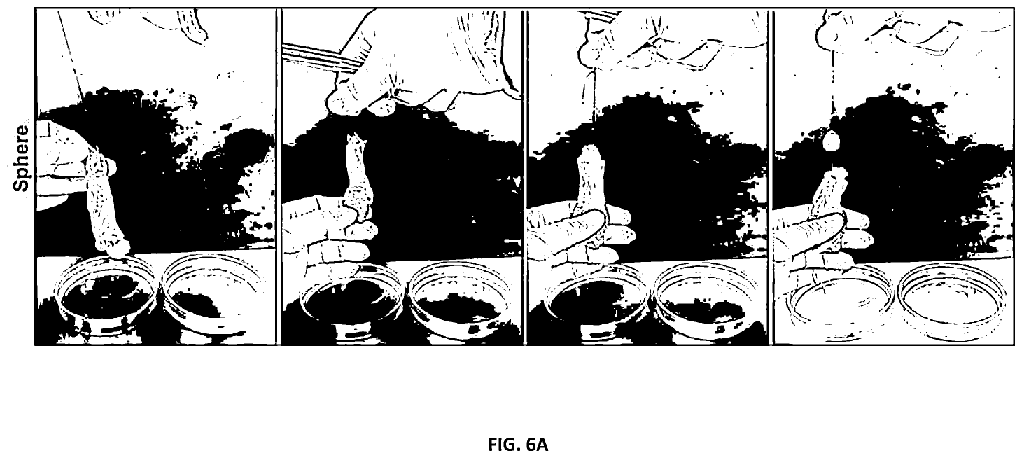

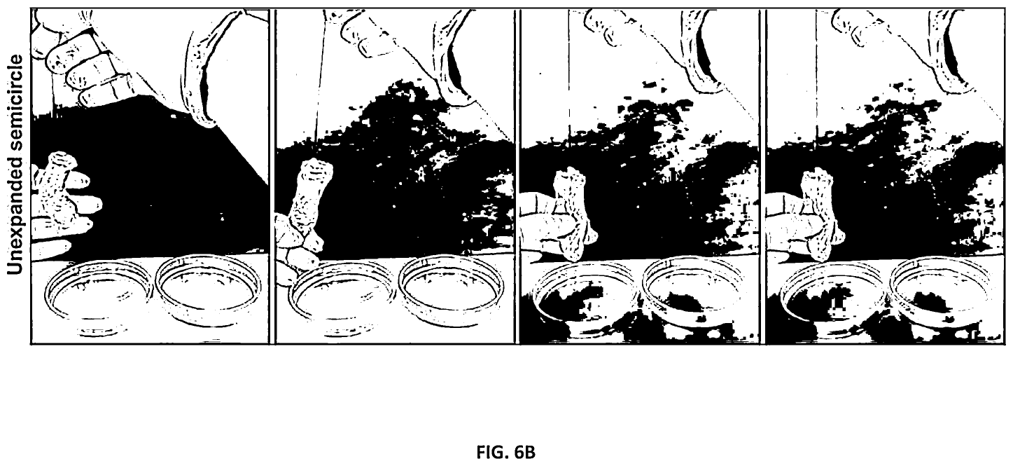

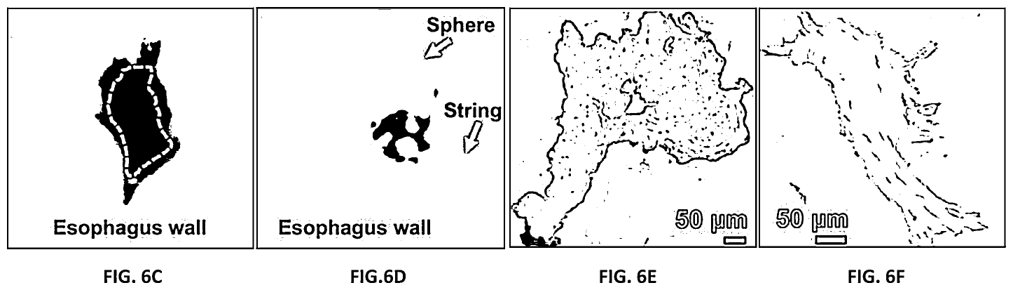

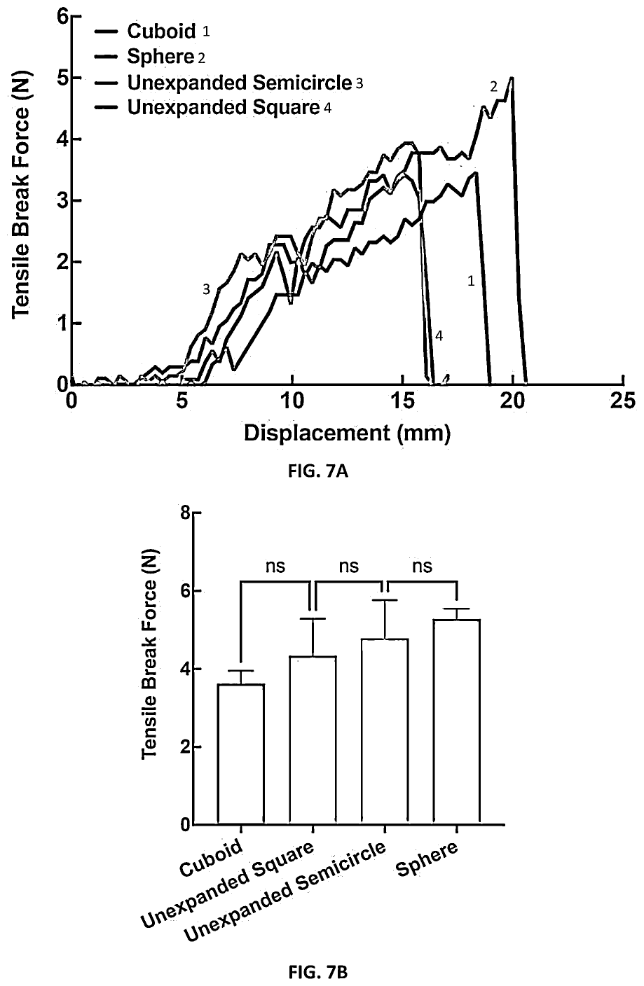

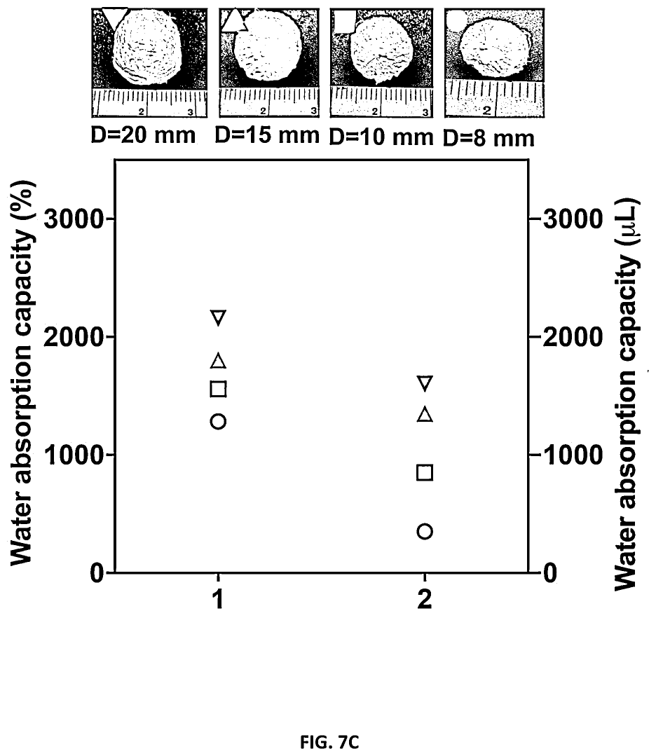

Barret esophagus (B.E.) is a serious disease in which abnormal cells develop in the lining of the esophagus (the digestive tube that connects the throat to the stomach) (Fitzgerald, et al., Lancet (2020) 396:333-344; Offman, et al., BMC Cancer (2018) 18:784; Benaglia, et al., Gastroenterology (2013) 144:62; di Pietro, et al., Gastroenterology (2015) 148:912-923). Individuals who have experienced long-term gastroesophageal reflux, commonly known as acid reflux or heartburn, are more likely to develop B.E. because chronic exposure to regurgitated stomach acids inflames the esophageal lining (Offman, et al., BMC Cancer (2018) 18:784). Generally, endoscopy and biopsy are the gold standards for identifying the B.E (Fitzgerald, et al., Lancet (2020) 396:333-344; Offman, et al., BMC Cancer (2018) 18:784; Benaglia, et al., Gastroenterology (2013) 144:62; di Pietro, et al., Gastroenterology (2015) 148:912-923). However, this procedure requires sedation, is expensive and uncomfortable, and may carry some risks in the internal organs. To demonstrate the potential of nanofiber capsules for sampling B.E., the nanofiber capsules were tested using an esophagus-mimicking polyethylene tube (d=2 cm). Cuboid- and sphere-shaped nanofiber objects and square and semicircular nanofiber mats can be pulled through the simulated esophagus tube, indicating that the cuboid- and sphere-shaped nanofiber objects and unexpanded nanofiber square and semicircular mats can be readily retrieved through the tube. In addition, the sphere- and cuboid-shaped nanofiber objects were able to fit tightly in the tubes, whereas unexpanded nanofiber mats fit loosely in the tubes. Since Cytosponge™ is probably associated with problem like the detachment of the string from the Cytosponge™ during the retrieval, the mechanical properties of the nanofiber capsule on a string was tested (Januszewicz, et al., Clin. Gastroenterol. Hepatol. (2019) 4:647). FIG. 7A represents the break force or the string detachment force from the different nanofiber objects. While comparing with other nanofiber objects, the sphere showed the highest break force (5.7±1 N) (FIG. 7B). A nanofiber sphere was pulled on a string through the porcine esophagus and performed H&E staining of the collected cells. FIGS. 6A and 6B show photographs illustrating the cell/tissue collection process from the porcine esophagus using both nanofiber spheres and mats. Nanofiber spheres passed through the porcine esophagus easier than unexpanded membranes. FIGS. 6C and 6D show the endoscopic images of the inner wall of porcine esophagus and the nanofiber microsphere inside of the porcine esophagus, respectively. The sphere adhered closely to the inner wall of the esophagus (FIG. 6D), which may help to collect cells and tissues on the inner wall of the esophagus during sampling. The measured maximum extraction forces for unexpanded nanofiber square membranes, unexpanded nanofiber semicircular membranes, and expanded nanofiber spheres were 0.85±0.10 N, 0.81±0.12 N, and 0.34±0.06 N, respectively. A larger force was used to pull the unexpanded nanofiber membranes because the corner of the unexpanded membranes was poking on the esophagus wall. Moreover, it may damage the esophagus inner wall rather than soft swabbing of cells. FIGS. 6E and 6F show the H&E staining of nanofiber spheres and mats after passing through the porcine esophagus explants. Spheres could collect epithelial cells and tissues from the inner wall of porcine esophagus. However, the unexpanded semicircular nanofiber membranes failed to collect any cells or tissues.

String test also called “Entero Test,” which consists of a gelatin capsule containing a long nylon string (e.g., 90 cm and 140 cm) with a weight attached to it, has been used to collect specimens from the upper part of the small intestine to detect parasites (Guiney, et al., Br. J. Clin. Pharmacol. (2011) 72:133-142; Arboleda, et al., J. Pediatr. Gastroenterol. Nutr. (2013) 57:192-196). These devices were also evaluated as a tool to collect bile secretion and gastric fluids for identifying metabolized drugs and pathogens (Guiney, et al., Br. J. Clin. Pharmacol. (2011) 72:133-142). It was reported that 1 cm of string could absorb 10 μl fluid (Guiney, et al., Br. J. Clin. Pharmacol. (2011) 72:133-142). In this study, to extend the possibilities of collecting samples from different sites like the duodenal site, different sized nanofiber spheres were designed by changing the radius of the semicircular mats before expansion. FIG. 7C shows photographs of different sizes of nanofiber spheres and their water absorption capacity. The 20 mm-diameter nanofiber spheres absorbed nearly 2100% (1600 μl) of its dry mass water. Decreasing the size of spheres decreased absorption capacity to 1700% (1350 μl) for 15-mm diameter spheres, to 1500% (850 μl) for 10-mm diameter spheres, and 1250% (350 μl) for 8-mm diameter spheres. While comparing with “Entero Test” with nylon string, the 8-mm diameter nanofiber spheres exhibited 35 times higher absorption capacity than the nylon string, which could enable higher collection yield, enhance testing sensitivity, and reduce false negative results. However, the nanofiber spheres could be contaminated with other biological fluids or tissues during the extraction. The nanofiber objects can be modified with certain antibodies for targeted collection of biological samples.

Electrospun nanofibers are used in many biomedical applications including drug delivery, tissue engineering, regenerative medicine, tissue modeling, biosensing, bioseparation, and biological specimen collection (Chen, et al., J. Mater. Chem. B (2020) 8:3733; Xue, et al., Acc. Chem. Res. (2017) 2050:1976-1987; Xue, et al., Chem. Rev. (2019) 119:5298-5415). Herein, a swallowable, re-expandable, ultra-absorbable, and retrievable nanofiber capsule is reported that was fabricated by attaching a string to a biocompatible and biodegradable 3D object packed into a gelatin capsule. This swallowable device shows the ability for sampling cells/tissue and fluids from the inner wall of esophagus and from gastric and duodenal sites. Recent progress towards identifying gastric mucosal abnormalities with narrow-band imaging, endocytoscopy, and confocal laser endomicroscopy has been made (Waddingham, et al., Frontline Gastroenterol. (2021) 12:322-331). However, collecting biological specimens from the internal organs, like the esophagus, in a minimally invasive manner remains a clinical challenge. Esophageal cancer is the 6th most common cause of cancer-related death in the world (Napier, et al., World J. Gastrointest. Oncol. (2014) 6:112-120). Endoscopic biopsy is the gold standard test for the diagnosis of esophageal cancer (Booth, et al., J. Gastrointest. Oncol. (2012) 3:232-242). To find an alternative of endoscopic BE screening, Cytosponge™, a single-use small device consisting of a small polyurethane sponge, about 30 mm in diameter, covered in a gelatin capsule, and attached to a string, was used for collection of cells from the lining of the esophagus in a minimally invasive method (Fitzgerald, et al., Lancet (2020) 396:333-344; Offman, et al., BMC Cancer (2018) 18:784; Benaglia, et al., Gastroenterology (2013) 144:62; di Pietro, et al., Gastroenterology (2015) 148:912-923). Despite progress in clinical trials, two reports were filed on the detachment of the removal string from the device during withdrawal from the patient's esophagus (Epelboym, et al., Oncologist (2014) 19:44-50). In this study, an alternative approach is reported using a biocompatible and biodegradable 3D nanofiber object on a string to collect the cells from the esophagus linings. Even if it detaches from the string, it can degrade in the acidic environment in the stomach. Moreover, if the sponges produce any small fragments due to frictions or detachment, the materials left over would not cause issues to the patients because of the biodegradability and biocompatibility of the materials. Gelatin capsules encasing sphere-shaped nanofiber objects on a string dissolve in water and re-expand to its original shape within 300 s (5 minutes), which is comparable to Cytosponge™ (Fitzgerald, et al., Lancet (2020) 396:333-344; Offman, et al., BMC Cancer (2018) 18:784; Benaglia, et al., Gastroenterology (2013) 144:62; di Pietro, et al., Gastroenterology (2015) 148:912-923; Katzka, et al., Clin. Gastroenterol. Hepatol., (2015) 13:77-83; Swart, et al., EclinicalMedicine (2021) 37:100969; Januszewicz, et al., Clin. Gastroenterol. Hepatol. (2019) 4:647). Importantly, after complete dissolution and re-expansion, the sphere-shaped nanofiber object can fit well with the contour of the inner wall of excised porcine esophagus explant during removal. Further, the histology analysis revealed that the epithelial cells/tissues were collected on sphere-shaped nanofiber objects after their retrieval from the explants. Although normal porcine esophagus was used for the demonstration, it is anticipated that the narrow esophagus in humans would demonstrate similar cell/tissue yields. Nanofiber capsules may also be tested using a large animal esophagus cancer model. Nanofiber capsules may also be tested for screening Barrett's esophagus patients in combination with biomarkers like trefoil factor 3 (Swart, et al., EclinicalMedicine (2021) 37:100969).

3D nanofiber matrices after transformation from 2D electrospun mats and gelatin-coating and crosslinking possess ultra-absorptive capability and super-elastic properties (Wang, et al., Clin. Cancer Res. (2019) 25:2127-2135; Jiang, et al., ACS Biomater. Sci. Eng. (2015) 1:991-1001; Chen, et al., Appl. Phys. Rev. (2020) 7:021406; Chen, et al., Nano Lett. (2019) 19:2059-2065; Chen, et al., Adv. Mater. (2020) 32:2003754). To enhance the hydrophilicity of the nanofibers, Pluronic® F127 was incorporated into the PCL nanofibers. In this study, biological samples were in the liquid except for esophagus tissues. The collection of biological samples was mainly attributed to liquid absorption rather than the interaction between the nanofibers and each microorganism. In addition, the collection of esophagus tissues was mainly attributed to the scratching of the inner wall of the esophagus during the pulling. Similarly, the sphere-shaped nanofiber objects generated in a similar approach were able to absorb a large amount of liquid (20 times their original mass) and return to their original shape after release from capsular compression in both liquid and air (Jiang, et al., ACS Biomater. Sci. Eng. (2015) 1:991-1001; Chen, et al., Appl. Phys. Rev. (2020) 7:021406; Chen, et al., Nano Lett. (2019) 19:2059-2065; Chen, et al., Adv. Mater. (2020) 32:2003754; McCarthy, et al., Nano Lett., (2021) 21:1508-1516). These properties allow for applications in sampling gastric fluids. Herein, a minimally invasive method is provided of using swallowable gelatin capsules encasing compressed nanofiber objects on strings for collecting gastric fluids. Sampling gastric fluids could be useful for gastric acid analysis which is currently conducted through nasogastric tube or catheter-based esophageal pH-monitoring for detecting gastric disorders including peptic ulcer disease, and Zollinger-Ellisson syndrome (Epelboym, et al., Oncologist (2014) 19:44-50). Sampling gastric juice could also be used for diagnosis of gastric cancer and tuberculosis (Chae, et al., Am. J. Clin. Pathol. (2013) 140:209-214; Lobato, et al., Pediatrics (1998) 102: E40). The 3D expandable nanofiber capsule on a string with high absorption capacity and an easy retrieval would be an appealing choice to collect gastric juice. In addition, such a capsule could be used for collection of duodenal bile or fluids in duodenum site after modulating its size (e.g., diameter <15 mm).

Pathogen sampling is often performed via oral cavity, throat, or other internal organs like the esophagus and stomach, where bacteria-containing droplets could be absorbed onto the sampling materials and identified by RT-PCR, morphological phenotyping, or quadrant streaking and culture. In this work, colony density was distinguishably higher in plates incubated with the cuboid- and sphere-shaped nanofiber objects in lower concentrations of 10³, 10⁴, and 10⁵ CFU/ml of MRSA, but not in plates incubated with unexpanded nanofiber membranes. In addition, 3D nanofiber objects exhibited reduced cycle thresholds at most titer concentrations except for 10¹ pfu/ml and identified SARS-COV-2 at 10° pfu/ml, a concentration ten times lower than the lowest identifiable titer using cotton or flocked swabs reported (McCarthy, et al., Nano Lett. 21 (2021) 21:1508-1516). During the ongoing COVID19 pandemic, the implementation of 3D nanofiber objects for sampling may be able to identify low titers of virus at the early stage of SARS-COV-2 infection. Increasing the sensitivity of testing allows for virus detection at earlier stages of infection. The ultrahigh absorptivity and release of biological materials and minimally invasive usage could make nanofiber capsules excellent candidates for improved pathogen sampling from the internal organs in a minimally invasive way. Certain ligands may be incorporated into the nanofiber sponge to collect specific analytes (e.g., cells, exosomes) from the gastric site/esophagus sit for the early detection of various diseases.

In summary, a capsule comprising a compressed nanofiber sphere on a string has been developed that is swallowable, re-expandable, ultra-absorptive, and easily retrievable. Such a capsule can be used to collect biological specimens from internal organs like the esophagus and stomach in a minimally invasive manner. The nanofiber capsule collects cells/tissue from the inner surface of porcine esophagus explants. Unlike Cytosponge™, the nanofiber capsule could also be used as a swab for sampling pathogens including bacteria and viruses in the esophagus and stomach due to its ultra-absorption properties. Moreover, expanded nanofiber objects can increase the diagnostic accuracy or detect pathogen earlier due to their high sensitivity. The nanofiber capsule developed herein offers a minimally invasive method for collection of various biological specimens from gastric, oropharyngeal, esophagus and duodenal sites, which will help the early detection and timely treatment of many diseases originated with these organs. Remote sensing may be combined with the nanofiber capsule for in situ monitoring pH, cytokines or other markers.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for producing a nanofiber collection device, said method comprising:
a) fixing at least one point of a nanofiber mat, wherein said nanofiber mat comprises electrospun nanofibers,
b) expanding the nanofiber mat after step a) by exposing the nanofiber mat to gas bubbles, thereby producing an expanded nanofiber structure, and
c) attaching the nanofiber mat or the expanded nanofiber structure to an end of a string, thereby producing said nanofiber collection device.

2. The method of claim 1, wherein said expanded nanofiber structure has a rounded geometry.

3. The method of claim 1, wherein said expanded nanofiber structure is cylindrical, conical, spherical, or dome shaped.

4. The method of claim 1, wherein said expanded nanofiber structure has a spherical geometry.

5. The method of claim 1, wherein said gas bubbles are generated as a product of a chemical reaction.

6. The method of claim 5, wherein said chemical reaction is the hydrolysis of sodium borohydride.

7. The method of claim 1, wherein step b) comprises exposing the nanofiber mat to a subcritical fluid and depressurizing.

8. The method of claim 7, wherein said subcritical fluid is subcritical CO₂.

9. The method of claim 1, wherein said nanofiber mat comprises a plurality of aligned nanofibers, random nanofibers, and/or entangled nanofibers.

10. The method of claim 1, further comprising synthesizing said nanofiber mat by electrospinning prior to step a).

11. The method of claim 1, further comprising cutting said nanofiber mat prior to step a) or b), optionally wherein said nanofiber mat is frozen prior to cutting.

12. The method of claim 1, wherein said nanofiber mat comprises polycaprolactone (PCL).

13. The method of claim 1, wherein said nanofiber mat comprises a poloxamer.

14. The method of claim 13, wherein said poloxamer is poloxamer 407.

15. The method of claim 1, wherein step a) comprises thermally fixing at least one point of said nanofiber mat.

16. The method of claim 1, wherein step a) comprises fixing at least an entire side of said nanofiber mat.

17. The method of claim 1, further comprising coating the expanded nanofiber structure with a hydrogel.

18. The method of claim 17, wherein said hydrogel comprises gelatin, gelatin methacryloyl(GelMA), and/or chitosan.

19. The method of claim 17, further comprising crosslinking the expanded nanofiber structure and/or hydrogel.

20. The method of claim 19, wherein said crosslinking comprises contacting said expanded nanofiber structure and/or hydrogel to glutaraldehyde.

21. The method of claim 1, wherein step c) comprises attaching the string to the expanded nanofiber structure with an adhesive.

22. The method of claim 1, wherein step c) comprises attaching the string to the expanded nanofiber structure by physical means.

23. The method of claim 1, wherein the string is attached to the fixed portion of the nanofiber mat of the expanded nanofiber structure.

24. The method of claim 1, further comprising encapsulating the expanded nanofiber structure in a capsule.

25. The method of claim 24, wherein said capsule comprises gelatin.

26. A nanofiber collection device produced by the method of claim 1.

27. A nanofiber collection device comprising an expanded nanofiber structure and a string, wherein said expanded nanofiber structure is attached to a terminus of said string.

28. The nanofiber collection device of claim 27, wherein said expanded nanofiber structure is encapsulated within a capsule.

29. A method of collecting a sample from a subject, said method comprising contacting a nanofiber collection device of claim 27 with a site in said subject, wherein the sample is collected from the site via the expanded nanofiber structure.

30. The method of claim 29, wherein the sample comprises a bacterium, a virus, a cell, a tissue, or a fluid.

31. The method of claim 30, wherein the sample comprises a nucleic acid or a polypeptide.

32. The method of claim 29, further comprising retrieving the expanded nanofiber structure from the subject by pulling said string.

33. The method of claim 29, further comprising analyzing the sample for the presence of a disease or disorder.

34. The method of claim 33, wherein said disease or disorder is selected from the group consisting of Barret Esophagus, acid reflux, SARS-COVID-19, Helicobacter pylori, and cancer.

35. The method of claim 33, wherein said disease or disorder is gastric cancer or esophageal cancer.
